(19) United States
(12) Patent Application Publication
(10) Pub. No.: US 2023/0141187 A1
(43) Pub. Date: May 11, 2023

(54) NON-HUMAN ANIMAL SECRETOME MODELS

(71) Applicant: University of Pittsburgh - Of the Commonwealth System of Higher Education, Pittsburgh, PA (US)

(72) Inventors: Jie Liu, Pittsburgh, PA (US); Toren Finkel, Pittsburgh, PA (US)

(21) Appl. No.: 17/917,992

(22) PCT Filed: Apr. 21, 2021
(86) PCT No.: PCT/US2021/028340

§ 371 Date: Oct. 10, 2022

Related U.S. Application Data
(60) Provisional application No. 63/013,453, filed on Apr. 21, 2020.

(51) Int. Cl. A01K 67/027; C12N 9/00

(52) U.S. Cl. CPC A01K 67/0275; C12Y 603/04015; C12N 9/93; A01K 2217/206; A01K 2267/0393; C07K 2319/02; C07K 2319/04; C07K 2319/40; A01K 2227/103; A01K 2217/15

(57) ABSTRACT

This document relates to methods and materials involved in the deconvolution of serum. For example, transgenic non-human animals (e g , transgenic mice) that secrete tagged (e.g., biotinylated) molecules from a particular tissue are provided.

### CROSS-REFERENCE TO RELATED APPLICATIONS

This application claims the benefit of U.S. Patent Application Ser. No. 63/013,453, filed on Apr. 21, 2020. The disclosure of the prior application is considered part of (and is incorporated by reference in) the disclosure of this application.

### TECHNICAL FIELD

This document relates to methods and materials involved in the deconvolution of serum and other biological fluids. For example, transgenic non-human animals (e.g., transgenic mice) that secrete tagged (e.g. biotinylated) molecules from a particular tissue are provided.

### BACKGROUND INFORMATION

Human plasma contains a complex mixture of thousands of proteins derived from multiple tissues. While the tissue origin of certain abundant proteins like albumin are known, the complete contribution of any given cell type to the plasma proteome currently remains difficult to elucidate. Such information would undoubtedly be useful, as there is a growing realization that the abundance of certain proteins might provide unique insight into human health (Lehallier et al., Nat Med.; 25:1843-1850 (2019); and Williams et al., Nat Med.; 25:1851-1857 (2019)).

### SUMMARY

This document relates to methods and materials involved in the deconvolution of secreted molecules (e.g., secreted polypeptides) and/or membrane molecules (e.g., membrane polypeptides) that are secreted or shed from cells. For example, this document provides transgenic non-human animals (e.g., transgenic mice) designed to produce tagged (e.g. biotinylated) polypeptides that are released from a particular cell type (or a tissue containing that particular cell type). In some cases, a transgenic non-human animal provided herein can be designed to release endothelial-specific tagged molecules (e.g., can be designed to release tagged polypeptides from endothelial cells). In some cases, a transgenic non-human animal provided herein can be designed to release muscle-specific tagged molecules (e.g., can be designed to release tagged polypeptides from myocytes). Transgenic non-human animals (e.g., transgenic mice) designed to release molecules (e.g., polypeptides) that are tagged (e.g., biotinylated) in a tissue-specific manner can be referred to transgenic tissue-specific secretome non-human animals (e.g., endothelial-specific secretome mice or muscle-specific secretome mice).

As described herein, proximity-dependent biotin identification (BioID) polypeptides that include (a) a promiscuous biotin ligase activity such as that derived from Escherichia coli BirA, (b) an endoplasmic reticulum (ER) retention signal, and optionally (c) a human influenza hemagglutinin (HA) tag (e.g., a polypeptide such as an ER-BioID polypeptide or an ER-BioID^{HA} polypeptide) can localize to the lumen of the ER in cells such that, in the presence of biotin, the cells expressing the polypeptide (e.g., the ER-BioID polypeptide or the ER-BioID^{HA} polypeptide) produce biotinylated polypeptides that can be released form the cell and readily identified as originating from those cells using streptavidin or avidin purification followed by amino acid analysis (e.g., mass spectroscopy). Also as described herein, in cases where a transgenic mouse is designed to express one or more ER-BioID polypeptides in only a particular cell type, that transgenic mouse can be used to capture an in vivo tissue-specific secretome. For example, a transgenic mouse expressing one or more ER-BioID^{HA} polypeptides in endothelial cells (but not other types of cells), can release (e.g., secrete) biotinylated polypeptides from endothelial cells, and the endothelial-specific secretome can be readily identified using, for example, streptavidin purification followed by mass spectroscopy. In some cases, a transgenic mouse expressing one or more ER-BioID^{HA} polypeptides in myocytes (but not other types of cells), can release (e.g., secrete) biotinylated polypeptides from myocytes, and the muscle-specific secretome can be readily identified using, for example, streptavidin purification followed by mass spectroscopy.

The ability to tag molecules released (e.g., secreted) from a particular tissue type (e.g., by expressing one or more polypeptides that localize to the ER lumen and have the ability to tag molecules within the ER such as ER-BioID or ER-BioID^{HA} in a particular cell type) provides a unique and unrealized opportunity to identify the cell or tissue origin of a released (e.g., secreted) polypeptide (e.g., a circulating secreted polypeptide). The identification of the cell or tissue origin of a released (e.g., secreted) polypeptide can be used to identify biomarkers associated with specific conditions (e.g., test conditions) such as stresses (e.g., physiological stresses such as exercise) or disease (e.g., the presence of a disease and/or progression of a disease) or disorder. In some cases, using released (e.g., secreted) polypeptides as biomarkers can allow clinicians to detect the biomarkers using non-invasive methods such as a liquid biopsy.

In general, one aspect of this document features a transgenic non-human animal, where the somatic cells of the non-human animal include nucleic acid having a nucleic acid sequence encoding a polypeptide including biotin ligase activity and an ER retention signal. The non-human animal can be a mouse. The ER retention signal can be KDEL (SEQ ID NO:1), SDEL (SEQ ID NO:6, YEEL (SEQ ID NO:7), or RDEL (SEQ ID NO:8). The biotin ligase activity can be a bacterial BirA biotin ligase activity. The polypeptide can include a peptide tag. The peptide tag can be an HA tag, a Myc-tag, a FLAG-tag a T7-tag, or a V5-tag. The polypeptide can include an amino acid sequence of a biotin ligase followed by the ER retention signal. The polypeptide can include an amino acid sequence of a biotin ligase followed by a peptide tag followed by the ER retention signal. The nucleic acid can include a promoter sequence upstream of the nucleic acid sequence. The nucleic acid can include a promoter sequence followed by a recombinase recognition site followed by an intervening nucleic acid sequence followed by a recombinase recognition site followed by the nucleic acid sequence, where expression of the polypeptide does not occur unless a recombinase excises the intervening nucleic acid sequence via the recombinase recognition sites. The intervening nucleic acid sequence can encode one or more stop codons. The intervening nucleic acid sequence can encode an intervening polypeptide. The intervening polypeptide can be an EGFP polypeptide. The expression of the polypeptide within a somatic cell of the non-human animal results in secreted or membrane polypeptides of the somatic cell being biotinylated in the presence of biotin. The non-human animal can be a non-human animal that consumed water, food, or both including biotin. The non-human animal can be heterozygous for the nucleic acid. The non-human animal can be homozygous for the nucleic acid. The somatic cells of the non-human animal can include a second nucleic acid, where the second nucleic acid can include a nucleic acid sequence encoding a polypeptide having recombinase activity. The polypeptide having recombinase activity can be a cre recombinase. The second nucleic acid can include a tissue-specific promoter sequence operably linked to the nucleic acid sequence encoding the polypeptide having the recombinase activity. The tissue-specific promoter sequence can be an endothelial cell-specific promoter sequence, a myocyte-specific promoter sequence, a pancreatic-specific promoter sequence, a kidney-specific promoter sequence, or an adipocyte-specific promoter sequence. The non-human animal can be heterozygous for the second nucleic acid. The non-human animal can be homozygous for the second nucleic acid.

In another aspect, this document features methods for making a transgenic non-human animal, where the somatic cells of the non-human animal include nucleic acid having a nucleic acid sequence encoding a polypeptide including biotin ligase activity and an ER retention signal. The methods can include, or consist essentially of, introducing the nucleic acid into a cell and allowing the cell to develop into the transgenic non-human animal. The cell can be an egg cell or a cell of an embryo.

In another aspect, this document features methods for making a transgenic non-human animal. The methods can include, or consist essentially of, mating a first mating partner with a second mating partner of the opposite sex, as compared to the first mating partner and of the same species as the first mating partner, to produce at least one offspring, where the first mating partner is a transgenic non-human animal, where the somatic cells of the non-human animal include nucleic acid having a nucleic acid sequence encoding a polypeptide including biotin ligase activity and an ER retention signal, and where the gamete of the first mating partner that results in the offspring includes the nucleic acid.

In another aspect, this document features methods for making a transgenic non-human animal. The methods can include, or consist essentially of, mating a first mating partner with a second mating partner of the opposite sex, as compared to the first mating partner and of the same species as the first mating partner, to produce at least one offspring, where the first mating partner is a first transgenic non-human animal, where the somatic cells and gametes of the first transgenic non-human animal include a first nucleic acid including a nucleic acid sequence encoding a polypeptide having biotin ligase activity and an ER retention signal, where the second mating partner is a second transgenic non-human animal, where the somatic cells and gametes of the second transgenic non-human animal include a second nucleic acid including a nucleic acid sequence encoding a polypeptide having recombinase activity, and where the gamete of the first mating partner that results in the offspring includes the first nucleic acid. The gamete of the second mating partner that results in the offspring can include the second nucleic acid. The first transgenic non-human animal can be a transgenic non-human animal where the somatic cells of the non-human animal include nucleic acid having a nucleic acid sequence encoding a polypeptide including biotin ligase activity and an ER retention signal. The polypeptide having recombinase activity can be a cre recombinase. The second nucleic acid can include a tissue-specific promoter sequence operably linked to the nucleic acid sequence encoding the polypeptide having the recombinase activity. The tissue-specific promoter sequence can be an endothelial cell-specific promoter sequence, a myocyte-specific promoter sequence, pancreatic-specific promoter sequence, a kidney-specific promoter sequence, or an adipocyte-specific promoter sequence. The second transgenic non-human animal can be heterozygous for the second nucleic acid. The second transgenic non-human animal can be homozygous for the second nucleic acid.

In another aspect, this document features a transgenic non-human animal, where the somatic cells of a cell type of the non-human animal includes nucleic acid including a promoter sequence operably linked to a nucleic acid sequence encoding a polypeptide having biotin ligase activity and an ER retention signal, where the somatic cells of the cell type express the polypeptide. The animal can be a mouse. The ER retention signal can be KDEL (SEQ ID NO:1), SDEL (SEQ ID NO:6, YEEL (SEQ ID NO:7), or RDEL (SEQ ID NO:8). The biotin ligase activity can be a bacterial BirA biotin ligase activity. The polypeptide can include a peptide tag. The peptide tag can be an HA tag, a Myc-tag, a FLAG-tag, a T7-tag, or a V5-tag. The polypeptide can include an amino acid sequence of a biotin ligase followed by the ER retention signal. The polypeptide can include an amino acid sequence of a biotin ligase followed by a peptide tag followed by the ER retention signal. The somatic cells of the non-human animal that are a different cell type from the cell type can include nucleic acid including a promoter sequence followed by a recombinase recognition site followed by an intervening nucleic acid sequence followed by a recombinase recognition site followed by a nucleic acid sequence encoding the polypeptide, where expression of the polypeptide does not occur in the somatic cells of the different cell type. The intervening nucleic acid sequence can encode one or more stop codons. The intervening nucleic acid sequence can encode an intervening polypeptide. The intervening polypeptide can be an EGFP polypeptide. The expression of the polypeptide within a somatic cell of the cell type can result in secreted or membrane polypeptides of the somatic cell being biotinylated in the presence of biotin. The non-human animal can be a non-human animal that consumed water, food, or both including biotin. The non-human animal can be heterozygous for the nucleic acid. The non-human animal can be homozygous for the nucleic acid. The somatic cells of the non-human animal can include a second nucleic acid, where the second nucleic acid can include a nucleic acid sequence encoding a polypeptide having recombinase activity. The polypeptide having recombinase activity can be a cre recombinase. The second nucleic acid can include a tissue-specific promoter sequence operably linked to the nucleic acid sequence encoding the polypeptide having the recombinase activity. The tissue-specific promoter sequence can be an endothelial cell-specific promoter sequence, a myocyte-specific promoter sequence, pancreatic-specific promoter sequence, a kidney-specific promoter sequence, or an adipocyte-specific promoter sequence. The non-human animal can be heterozygous for the second nucleic acid. The non-human animal can be homozygous for the second nucleic acid.

In another aspect, this document features methods for identifying a secreted or membrane polypeptide expressed by a cell type within a transgenic non-human animal, where the somatic cells of the cell type of the non-human animal include nucleic acid including a promoter sequence operably linked to a nucleic acid sequence encoding a polypeptide including biotin ligase activity and an ER retention signal, where the somatic cells of the cell type express the polypeptide, where the transgenic non-human animal was treated with biotin, where the secreted or membrane polypeptide expressed by the cell type becomes biotinylated via the polypeptide, thereby forming a biotinylated polypeptide of the cell type. The methods can include, or consist essentially of, (a) isolating the biotinylated polypeptide from a sample obtained from the transgenic non-human animal, and (b) identifying the biotinylated polypeptide. The sample can be a plasma sample. The isolating step can include capturing the biotinylated polypeptide via binding to streptavidin. The identifying step can include amino acid sequence analysis of the isolated biotinylated polypeptide. The identifying step can include performing mass spectroscopy. The transgenic non-human animal can have been exposed to a test condition or can have a disease or disorder. The method can include identifying the biotinylated polypeptide as being overexpressed or underexpressed as a result of the test condition, disease, or disorder. The transgenic non-human animal can have been exposed to a test condition. The test condition can be an exercise test condition or diet test condition. The transgenic non-human animal can have a disease or disorder. The disease or disorder can be a cancer, an autoimmune condition, heart disease, a neurological disorder, an endocrine disorder, kidney disease, pancreatic disease, or stroke.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E: Expression of ER-BioID^{HA}allows for the rapid purification of secreted polypeptides from endothelial cells. FIG. 1A) Diagram of an exemplary ER-BioID^{HA} construct containing a C-terminal KDEL ER retention sequence (SEQ ID NO:1). FIG. 1B) Expression of ER-BioID^{HA} in endothelial cells co-localized with the ER protein calnexin. FIG. 1C) Detection of biotinylated polypeptides in endothelial cell conditioned media. Primary endothelial cells were infected with a control GFP lentivirus, or a lentivirus encoding ER-BioID^{HA}. Cells were exposed to biotin (50 μM) where indicated. Total supernatant was purified with streptavidin (SA) beads and then analyzed by Western blot using SA. FIG. 1D) Secreted polypeptides identified from ER-BioID^{HA}-expressing endothelial cells under basal condition or 3 days after being stimulated to undergo EndoMT. Numbers represent peptide counts recovered. FIG. 1E) Analysis of endothelial cell lysate under basal conditions (-) or 3 days after EndoMT induction. Three hits identified were assessed, and tubulin is shown as a loading control.

FIGS. 2A-2E: Generation and characterization of an exemplary secretome mouse. FIG. 2A) Diagram of an exemplary floxed transgene. Cre expression leads to tissue-specific expression of ER-BioID^{HA}. Also shown is an exemplary work flow for the identification of cell or tissue-specific secretion. FIG. 2B) Endothelial cell in vivo secretion identified by endothelial cell restricted ER-BioID^{HA} expression using VE-cadherin Cre. Mice possessing ER-BioID^{HA} but lacking Cre expression were used as a control. FIG. 2C) Tissue restricted expression of ER-BioID^{HA} was confined to skeletal muscle and heart in MCK-Cre positive mice. FIG. 2D) Skeletal muscle cell lysate in mice fed biotin with the indicated genotype. Multiple biotinylated polypeptides were evident in ER-BioID^{HA}/MCK-Cre animals. FIG. 2E) Analysis of serum for muscle-specific polypeptide abundance in ER-BioID^{HA}/MCK-Cre mice with or without one month of exercise. Peptide counts from mice expressing MCK-Cre but lacking ER-BioID^{HA} were used as a specificity control.

FIGS. 3A-3B: An exemplary biotin ligase. FIG. 3A) An amino acid sequence of a biotin ligase (SEQ ID NO:2). FIG. 3B) A nucleic acid sequence encoding a biotin ligase (SEQ ID NO:3).

FIGS. 4A-4B: An exemplary polypeptide that includes an ER retention signal and a biotin ligase. FIG. 4A) An amino acid sequence of a polypeptide that includes an IgK signal peptide (underlined), a biotin ligase, an HA tag ( [symbol] ), and an ER retention signal ( [symbol] [symbol] ) (SEQ ID NO:4). FIG. 4B) A nucleic acid sequence encoding a polypeptide that includes an IgK signal peptide (underlined), a biotin ligase, an HA tag ( [symbol] ), and an ER retention signal ( [symbol] [symbol] ) (SEQ ID NO:5).

FIGS. 5A-5E. Expression of ER-BioID^{HA} allows for the rapid purification of secreted proteins from endothelial cells. FIG. 5A) Diagram of the ER-BioID^{HA} construct. FIG. 5B) Expression of ER-BioID^{HA} in endothelial cells colocalizes with the ER resident protein calnexin. Primary endothelial cells were infected with a lentivirus encoding ER-BioID^{HA} or the pLJM1 empty vector lentivirus (−). Expression of BioID was determined using the HA-epitope with 4′,6-diamidino-2-phenylindole (DAPI) used as a nuclear counterstain. FIG. 5C) Detection of biotinylated proteins using streptavidin (SA)-based immunohistochemistry in endothelial cell-expressing ER-BioID^{HA} in the presence (50 μM) or absence of exogenous biotin. FIG. 5D) Detection of secreted biotinylated proteins using SA-based purification and SA-based Western blotting. Conditioned media of endothelial cells expressing ER-BioID^{HA} or GFP in the presence or absence of exogenous biotin was assessed. Initial cell lysate was assessed for ER-BioID^{HA} expression (HA epitope) and tubulin as a loading control. FIG. 5E) ER-BioID^{HA} protein was not detected in the supernatant (media) of ER-BioID^{HA} or GFP-expressing cells as assessed by HA-epitope assessment. Protein lysate from endothelial cells expressing ER-BioID^{HA} serve as a positive control.

FIGS. 6A-6D. ER-BioID^{HA} expression provides for a simple method to assess differential secretome of cells in culture. FIG. 6A) PCA analysis distinguishes the secretome of endothelial cells obtained at baseline compared to the secretome of endothelial cells 3 days after induction of EndoMT. Analysis was conducted using five technical replicates per condition. FIG. 6B) Secreted proteins identified as being increased following EndoMT. Proteins are ranked by their fold increase over baseline based on peptide counts recovered. FIG. 6C) Enzyme-linked immunosorbent assay (ELISA)-based determination of the level of three proteins previously determined by the ERBioID^{HA}—based strategy to increase following EndoMT induction (n=4-6 replicates per condition). FIG. 6D) Lysine content (percentage of total protein) for secreted endothelial proteins identified by the ER-BioID^{HA}—based strategy compared to a reference (Ref) endothelial cell secretome.

FIGS. 7A-7F. Generation and characterization of the secretome mouse. FIG. 7A) Diagram of the floxed transgene used. Cre expression leads to tissue-specific expression of ER-BioIDHA. FIG. 7B) Primary endothelial cells isolated from control mice (VE-cadherin Cre only) or experimental mice (ER-BioID^{HA}/VE-cadherin Cre). Evidence of transgene expression is only seen in cells isolated from the experimental mice as determined by HA-epitope staining. ER localization is evident using calnexin as an ER-specific marker. FIG. 7C) Biotinylated proteins as evident by SA staining are observed in endothelial cells obtained from the experimental but not control mice. FIG. 7D) Sparse partial least squares discriminant analysis (sPLS-DA) distinguishes the secretome of control mice (ER-BioID^{HA} only or Cre only) from the secretomeENDO experimental animals (n=4 mice per genotype). FIG. 7E) Purified serum proteins with the greatest VIP coefficients between control and experimental mice are shown. FIG. 7F) Western blot analysis of the serum of control and experimental mice with or without biotin supplementation is shown for two proteins with high VIP scores. These proteins are only identified in the setting ER-BioID^{HA} expression and biotin supplementation.

FIGS. 8A-8G. Analysis of the secretome^{MUSCLE} mice. FIG. 8A) Tissue-restricted expression of ER-BioIDHA is confined to skeletal muscle and heart in MCK-Cre positive mice. FIG. 8B) Skeletal muscle-derived lysate from pairs of mice fed biotin with the indicated genotype and assessed using SA detection. Skeletal muscle biotinylation is only observed in experimental animals expressing both MCK-Cre and ER-BioID^{HA}. Within tissues due to Cre-mediated excision HA-expression and GFP expression are inversely related. Tubulin is shown as a loading control. FIG. 8C) Biotinylation in skeletal muscle in mice where biotin is administered by intraperitoneal injection (i.p.), subcutaneous injection (SC), or mixed into the food for 2 days (chow) or by a combination of all three routes of administration (all). Tubulin is shown as a loading control. FIG. 8D) Analysis of control or experimental mice given biotin in their chow for 2 or 5 days. Tubulin is shown as a loading control. FIG. 8E) sPLS-DA distinguishes the secretome of secretome^{MSCLE} mice with or without exercise (n=4 mice per exercise condition) from each other and from control mice (Cre-recombinase only; n=7). FIG. 8F) Purified serum proteins with the greatest VIP coefficients between control and experimental mice are shown. A heat map of the relative abundance of these VIP proteins with and without exercise is shown. FIG. 8G) ELISA-based serum determination of myostatin, the VIP protein with the greatest coefficient, is shown using a separate group of wild-type mice with or without exercise.

FIG. 9. Loading control for endothelial supernatants. Supernatant used for FIG. 5D was assessed by Coomassie blue gel staining to ensure equal loading.

FIG. 10. Western blot analysis of endothelial cell lysate under basal conditions (−) or 3 days after EndoMT induction. Pentraxin-related protein 3 (PTX3) identified by ER-BioIDHA analysis and by ELISA-based methods was also assessed intracellularly. The induction of EndoMT results in marked PTX3 induction. Tubulin is shown as a loading control.

FIG. 11. Distribution of lysine residue content in endothelial secreted proteins. A previous analysis had identified 182 proteins secreted by endothelial cells in culture. The lysine content in these 182 proteins was analyzed and compared that to the top 182 proteins identified in the endothelial secretome of ER-BioIDHA-expressing endothelial cells. There is no evidence that ER-BioID identified proteins contain a higher percentage of lysine residues.

FIG. 12. Loading control for serum of Secretome mice or controls. Supernatant used for FIG. 7F was assessed by Coomassie blue gel staining to ensure equal loading.

FIGS. 13A-13B. Exercise regime results in change in body weight but not muscle weight. FIG. 13A) Effects of voluntary exercise regime on body weight. Animals were provided access to a running wheel (exercise) or not (−) for 4 weeks. The change in body weight either absolute or percentage is shown for the two groups (n=4-6 mice per group). FIG. 13B) The weight of individual muscles including gastrocnemius (GAS), tibialis anterior (TA), soleus (SA) and extensor digitorum longus (EDL) is shown. Exercise was of sufficient intensity to alter body weight but not induce measurable hypertrophy.

FIG. 14. ER-BioID^{HA} protein is not secreted into the serum. No evidence of circulating ER-BioID^{HA} as assessed by HA-epitope assessment of the serum of control or experimental mice (n=3 per genotype). Protein lysate from muscle lysate of experimental mice serve as a positive control.

FIGS. 15A-15E. ER-targeted BioID2 to label secreted proteins. FIG. 15A) Schematic of lentivirus expressing ER-targeted BioID2 construct. FIG. 15B) Photomicrographs of A549 and A549-BioID2 cells showing co-localization of the BioID2 (HA-tagged; green) and the ER marker calnexin (red). Nuclei are stained with DAPI (blue). Scale bar is 25 microns. FIG. 15C) Proof of concept demonstrating that secreted proteins are biotinylated by ER-targeted BioID2. A549 or A549 cells stable expression ER-BioID2 were transfected with a plasmid encoding a V5-SFTPA2 construct. Eighteen hours after transfection, biotin was added to some samples and the media and cell lysates were examined the next day by western blotting for SFTPA2 (V5), BioID2 (HA), or biotinylated proteins (Strep-HRP). GAPDH was a load control for cell lysates. Biotinylated proteins were detected in the media only in cells that expressed ER-BioID2 and were cultured in excess biotin. Biotinylated SFTPA2 was detected in media only when ER-BioID2 was present. FIG. 15D) Experimental procedure for unbiased proteomic analysis of secreted proteins. Cells were cultured for 5 days in the presence of Dox to induce senescence followed by incubation with excess biotin for 8 hours. After biotin labeling, cells were washed and fresh media was added. Twenty-four hours later, media was collected and biotinylated proteins were isolated by incubation with streptavidin-coated beads and analyzed by stain-free SDS-page (FIG. 15E).

FIGS. 16A-16E. Unbiased proteomic characterization of senescent secretome. FIG. 16A) Volcano plot showing relevant abundance of proteins in supernatant from TRF2-DN+Dox compared to TRF2-DN cells. The 10 most significantly upregulated (FIG. 16B) and downregulated (FIG. 16C) proteins between TRF2-DN (control) and TRF2-DN+Dox (senescent) cells (data are from a single experiment). FIG. 16D) Ingenuity Canonical Pathway analysis of differentially expressed secreted proteins shows and enrichment in pathways associated with coagulation, non-specific defense, adhesion, and lipid metabolism. FIG. 16E) Correlation of the fold-changes of RNA and protein from RNA-seq and proteomic data shows limited correlation between the two datasets.

FIGS. 17A-17C. Identification of polypeptides secreted from cultured organ. FIG. 17A: Bones harvested from neonatal mice can be maintained in culture. The top panel includes images of cultured bone. The bottom left graph shows a growth curve of cultured bone. The bottom right graph shows a calcification curve of cultured bone. FIG. 17B: A western blot for streptavidin showing that tagged molecule are secreted from cultured bone into the culture medium. FIG. 17C: An exemplary timeline for the experimental culture methods.

FIG. 28. A schematic of an exemplary method for identifying polypeptides secreted from pancreatic cancer. Serum collected from a transgenic PDX1-Cre is contacted with biotin. Biotinylated polypeptides secreted from the pancreas are isolated using streptavidin magnetics beads and identified using mass spectrometry.

### DETAILED DESCRIPTION

This document provides methods and materials involved in the deconvolution of released molecules (e.g., secreted polypeptides) within samples (e.g., blood samples) that contain molecules from various different sources (e.g., two or more different cell types). For example, this document provides transgenic non-human animals (e.g., transgenic mice) that release (e.g., secrete) tagged (e.g. biotinylated) polypeptides from a particular cell type (or a tissue containing that particular cell type). In some cases, a transgenic non-human animal provided herein can release (e.g., secrete) endothelial-specific tagged molecules (e.g., can secrete tagged molecules from tissues containing endothelial cells). In some cases, a transgenic non-human animal provided herein can secrete muscle-specific tagged molecules (e.g., can secrete tagged molecules from tissues containing myocytes).

A transgenic non-human animal described herein can release (e.g., secrete) any appropriate type of tagged molecule. Examples of molecules that can be tagged and released by particular cell types of a transgenic non-human animal described herein include, without limitation, polypeptides.

A tagged molecule (e.g., a tagged polypeptide) can be released (e.g., secreted or shed) from any type of cell (or a tissue containing the cell type) within the body of a transgenic non-human animal described herein (e.g., a transgenic non-human animal that secretes tissue-specific tagged molecules). Examples of cell types that can secrete or shed tagged molecules in a transgenic non-human animal described herein include, without limitation, endothelial cells, myocytes (e.g., cardiomyocytes), kidney cells, neurons (e.g., astrocytes and oligodendrocytes), pancreatic cells, osteocytes, and hematopoietic cells. In some cases, a single type of cell (or a tissue containing the cell type) within the body of a transgenic non-human animal described herein can release (e.g., secrete) a tagged molecule. In some cases, two or more (e.g., two, three, four, or more) types of cells (or tissues containing the cell types) within the body of a transgenic non-human animal described herein can release (e.g., secrete) a tagged molecule. In some cases, all types of cells within the body of a transgenic non-human animal described herein can release (e.g., secrete) a tagged molecule.

A tagged molecule (e.g., a tagged polypeptide) can be released (e.g., secreted or shed) into any appropriate location within the body of a transgenic non-human animal described herein (e.g., a transgenic non-human animal that secretes tissue-specific tagged molecules). In some cases, a tagged molecule can be released (e.g., secreted or shed) into an extracellular space (e.g., the extracellular matrix). In some cases, a tagged molecule can be released (e.g., secreted or shed) into an extracellular fluid (e.g., blood plasma, interstitial fluid, urine, and cerebrospinal fluid (CSF)). For example, a tagged molecule that is released (e.g., secreted or shed) into blood plasma can enter the bloodstream and can be referred to as a circulating secreted or shed molecule.

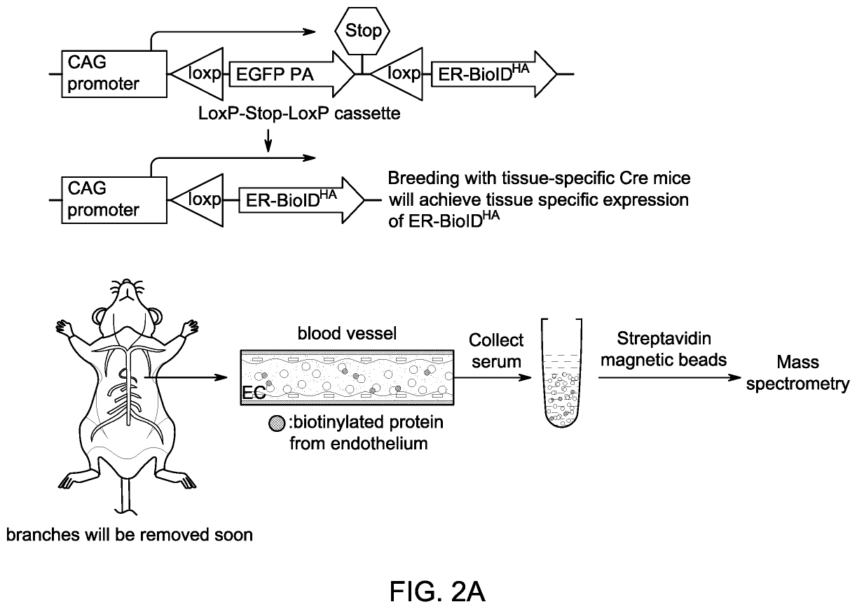

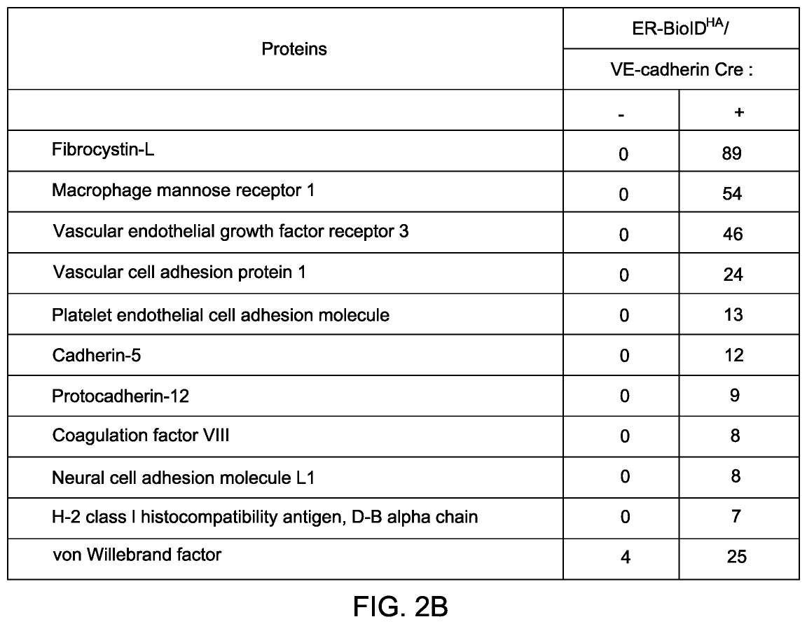

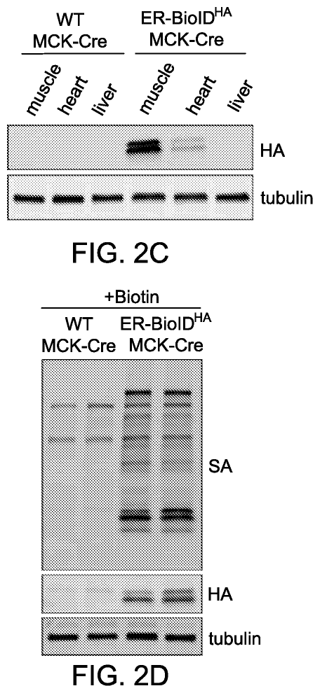

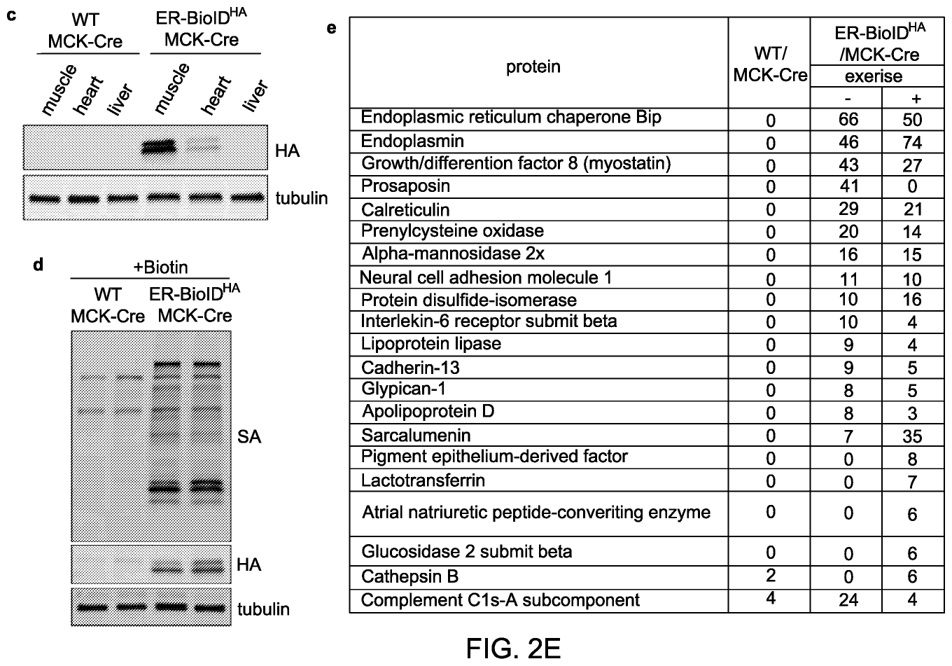

A transgenic non-human animal described herein can have (e.g., can be engineered to have) somatic cells and gametes with genomes that contain nucleic acid (e.g., exogenous nucleic acid) that encodes a polypeptide having ligase activity (e.g., biotin ligase activity) and having an ER retention signal. For example, a transgenic non-human animal described herein can include a transgene that encodes an ER-BioID polypeptide or an ER-BioID^{HA} polypeptide. In some cases, the somatic cells and gametes of such a transgenic non-human animal can contain the nucleic acid (e.g., exogenous nucleic acid) that encodes the polypeptide having ligase activity (e.g., biotin ligase activity) and having an ER retention signal in a manner such that the polypeptide is not expressed in any cells of the transgenic non-human animal. For example, the somatic cells and gametes of a transgenic non-human animal described herein can include nucleic acid (e.g., exogenous nucleic acid) that encodes the polypeptide having ligase activity (e.g., biotin ligase activity) and having an ER retention signal such that the polypeptide-encoding sequence is not operably linked to a promoter sequence, and instead includes an intervening nucleic acid sequence (e.g., a sequence including one or more stop codons) that needs to be removed in order to have a promoter sequence operably linked to that polypeptide-encoding sequence. Such an arrangement can be as shown in FIG. 2A (top diagram), in which case the intervening nucleic acid sequence located between the loxP sites needs to be removed before the CAG promoter sequence is operably linked to the ER-BioID^{HA} polypeptide-encoding nucleic acid sequence.

In some cases, a transgenic non-human animal provided herein where the somatic cells and gametes contain genomes having nucleic acid (e.g., exogenous nucleic acid) that encodes a polypeptide having ligase activity (e.g., biotin ligase activity) and having an ER retention signal in a manner such that the polypeptide is not expressed in any cells of the transgenic non-human animal can be used to generate a transgenic non-human animal where a particular selected cell type of that generated transgenic non-human animal expresses that polypeptide from the exogenous nucleic acid (or transgene). For example, a transgenic mouse line that contains a transgene, in the genome of its somatic cells and gametes, that includes a promoter sequence, followed by a first recombination site (e.g., loxP site), followed by intervening nucleic acid sequence that includes one or more stop codons, followed by a second recombination site (e.g., a lox P site), followed by a nucleic acid sequence encoding an ER-BioID polypeptide or an ER-BioID^{HA} polypeptide can be crossed with a transgenic mouse line that expresses a recombinase for those two recombination sites (e.g., cre recombinase) in a tissue-specific or cell-specific manner. The offspring of such a cross can have those tissues or cell types that express the recombinase also expressing the encoded polypeptide (e.g., the ER-BioID polypeptide or the ER-BioID^{HA} polypeptide) with the tissues or cell types that do not express the recombinase also not expressing the encoded polypeptide. The genetic arrangements of an offspring of such a cross can be as depicted in FIG. 2A with tissues/cells expressing the recombinase having the second from the top arrangement and with tissues/cells not expressing the recombinase having the top arrangement.

A transgenic non-human animal described herein (e.g., a transgenic non-human animal that secretes tissue-specific tagged molecules) can be any appropriate type of non-human animal. In some cases, a transgenic non-human animal provided herein can be a transgenic non-human mammal. Examples of non-human animals that can be used to generate a transgenic non-human animal described herein include, without limitation, zebrafish, Drosophila melanogaster, mice, rats, rabbits, guinea pigs, dogs, cats, pigs, sheep, horses, bovine species, and non-human primates (e.g., monkeys). In some cases, a transgenic non-human animal described herein can be a transgenic mouse.

In cases where a transgenic non-human animal described herein is a mouse, the mouse can be any appropriate type (e.g., strain) of mouse. Examples of strains of mice that can be used to make a transgenic non-human animal described herein include, without limitation, C57BL/6, BALB/c, FVB/N, and 129S1/S. In some cases, strains of mice that can be used to make a transgenic non-human animal described herein can include, without limitation, those set for in jax.org/jax-mice-and-services/find-and-order-jax-mice/most-popular-jax-mice-strains. In some cases, a mouse that can be used as (or can be used to generate) a transgenic non-human animal described herein can be an inbred strain of mouse. In some cases, a mouse that can be used as (or can be used to generate) a transgenic non-human animal described herein can be a hybrid strain of mouse.

A polypeptide having ligase activity (e.g., biotin ligase activity) and having an ER retention signal can include (e.g., can be engineered to include) any appropriate ER retention signal. An ER retention signal can be any appropriate signal that localizes a polypeptide that includes an ER retention signal and ligase activity (e.g., biotin ligase activity) to the ER. In some cases, an ER retention signal can retain a polypeptide that includes an ER retention signal and ligase activity (e.g., biotin ligase activity) within the ER lumen. Examples of ER retention signals that can be included in a polypeptide that includes an ER retention signal and ligase activity (e.g., biotin ligase activity) include, without limitation, a polypeptide having the amino acid sequence KDEL (SEQ ID NO:1), SDEL (SEQ ID NO:6, YEEL (SEQ ID NO:7), and RDEL (SEQ ID NO:8). In some cases, an ER retention signal can be as mpmp.huji.ac.il/maps/protER.html. For example, a nucleic acid (e.g., a transgene) that encodes a polypeptide having ligase activity (e.g., biotin ligase activity) and having an ER retention signal such that the polypeptide-encoding sequence is not operably linked to a promoter sequence, and instead includes an intervening nucleic acid sequence (e.g., a sequence including one or more stop codons) can include nucleic acid encoding an ER retention signal.

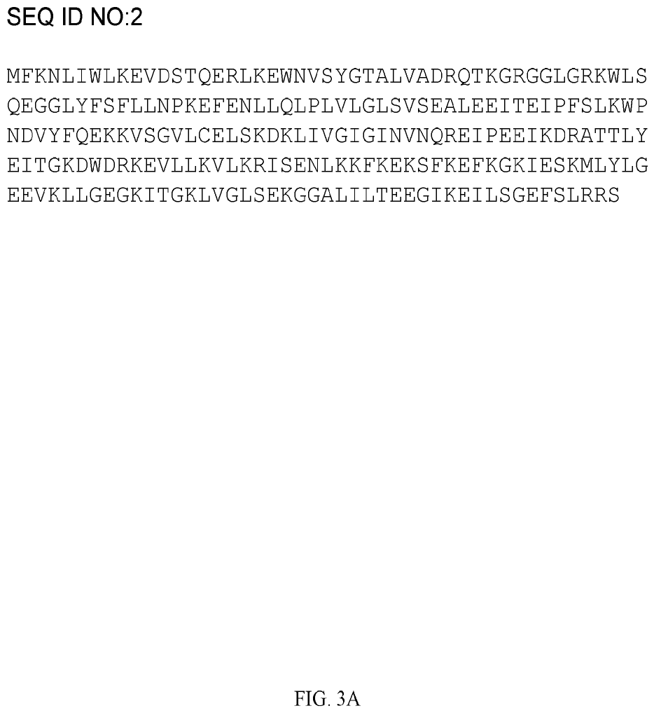

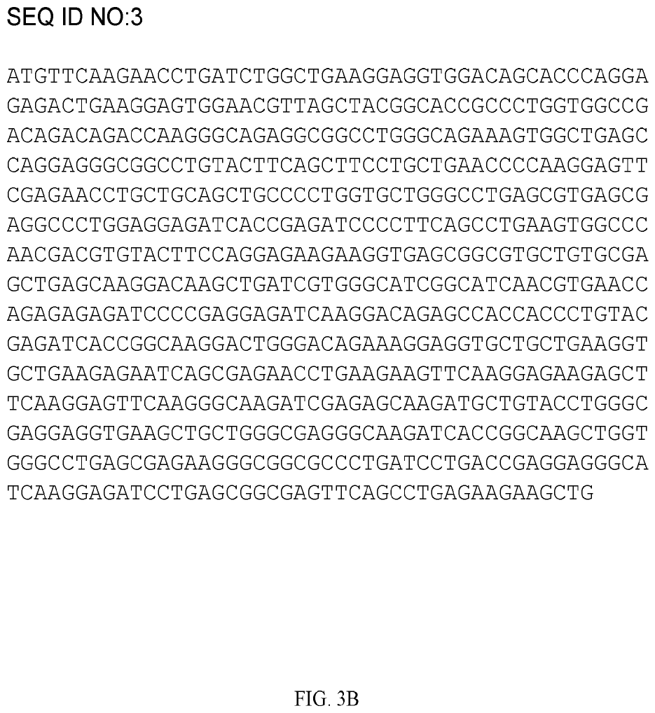

A polypeptide having ligase activity (e.g., biotin ligase activity) and having an ER retention signal can include (e.g., can be engineered to include) any appropriate ligase activity. In some cases, the ligase activity can add a tag to a molecule via an enzymatic reaction. In some cases, the ligase activity can covalently add a tag to a molecule. The ligase activity can add a tag to any appropriate part of a molecule (e.g., the N-terminus, the C-terminus, or an internal location). The ligase activity can be from any appropriate source. For example, the ligase activity can be a ligase activity of a naturally occurring polypeptide (e.g., a bacterial polypeptide such as an Escherichia coli polypeptide) or a recombinant ligase. Examples of ligases that can be used as described herein to tag one or more molecules include, without limitation, biotin ligases such as a BirA polypeptide, BioID, and BioID2. In some cases, a ligase can be as described elsewhere (see, e.g., Kim et al., Mol Biol Cell 27:1188-1196 (2016); and Branon et al., Nat Biotechnol. 36(9):880-887 (2018)). When the ligase activity is a biotin ligase activity, the biotin ligase activity can be from any appropriate biotin ligase. In some cases, a biotin ligase can include the amino acid sequence set forth in FIG. 3A. In some cases, a biotin ligase can be as described elsewhere (see, e.g., Kim et al., Trends Cell Biol.; 26:804-817 (2016); and Kim et al., Proc Natl Acad Sci USA; 111:E2453-61 (2014)). For example, a nucleic acid (e.g., a transgene) that encodes a polypeptide having ligase activity (e.g., biotin ligase activity) and having an ER retention signal such that the polypeptide-encoding sequence is not operably linked to a promoter sequence, and instead includes an intervening nucleic acid sequence (e.g., a sequence including a stop codon) can include nucleic acid encoding a ligase. When a ligase is a biotin ligase, a nucleic acid sequence that can encode the biotin ligase can be as set forth in FIG. 3B.

A polypeptide having ligase activity (e.g., biotin ligase activity) and having an ER retention signal can tag one or more molecules with any appropriate tag. In some cases, a tag can be an affinity tag. Examples of tags that can be added to one or more molecules include, without limitation, biotin.

A polypeptide having ligase activity (e.g., biotin ligase activity) and having an ER retention signal can tag a molecule with any appropriate number of tags. In some cases, a polypeptide having ligase activity and having an ER retention signal can add at least one (e.g., 1, 2, 3, 4, 5, 6, 7 or more) tag to a molecule. In some cases, a polypeptide having ligase activity and having an ER retention signal can add a plurality (e.g., 2, 3, 4, 5, 6, 7, or more) of tags to a molecule. For example, when a polypeptide having ligase activity and having an ER retention signal includes a biotin ligase, the biotin ligase can add at least one biotin to a molecule (e.g., a polypeptide) within the ER.

In some cases, a polypeptide having ligase activity (e.g., biotin ligase activity) and having an ER retention signal can be proximity-dependent. For example, a polypeptide having ligase activity (e.g., biotin ligase activity) and having an ER retention signal can tag one or more molecules (e.g., polypeptides) within the ER. For example, a polypeptide having ligase activity (e.g., biotin ligase activity) and having an ER retention signal can tag one or more molecules (e.g., polypeptides) within from about 0 nanometers (nm) to about 10 nm of the polypeptide having ligase activity (e.g., biotin ligase activity) and having an ER retention signal.

In some cases, one or more tags that can be added to a molecule (e.g., a polypeptide) by a polypeptide having ligase activity (e.g., biotin ligase activity) and having an ER retention signal can be provided to a non-human animal described herein (e.g., a transgenic non-human animal that secretes tissue-specific tagged molecules). For example, when a polypeptide having ligase activity (e.g., biotin ligase activity) and having an ER retention signal includes a biotin ligase, biotin can be provided to a non-human animal described herein. In some cases, when a non-human animal described herein where the somatic cells and gametes contain genomes that contain nucleic acid (e.g., exogenous nucleic acid) that encodes a polypeptide having biotin ligase activity and having an ER retention signal operably linked to a promoter sequence, the non-human animal can be administered biotin. Biotin can be administered to a non-human animal described herein using any appropriate method. In some cases, biotin can be administered to a non-human animal by injection (e.g., intraperitoneal (I. P.) injection and subcutaneous injection). In some cases, biotin can be administered to a non-human animal by consumption (e.g., by providing the non-human animal food and/or water containing biotin). Any appropriate amount of biotin can be administered to a non-human animal described herein. In some cases, from about 30 mg per kg body weight (mg/kg) to about 60 mg/kg biotin (e.g., from about 30 mg/kg to about 60 mg/kg biotin, from about 30 mg/kg to about 55 mg/kg biotin, from about 30 mg/kg to about 50 mg/kg biotin, from about 30 mg/kg to about 45 mg/kg biotin, from about 30 mg/kg to about 40 mg/kg biotin, from about 30 mg/kg to about 35 mg/kg biotin, from about 35 mg/kg to about 60 mg/kg biotin, from about 40 mg/kg to about 60 mg/kg biotin, from about 45 mg/kg to about 60 mg/kg biotin, from about 50 mg/kg to about 60 mg/kg biotin, from about 55 mg/kg to about 60 mg/kg biotin, from about 35 mg/kg to about 55 mg/kg biotin, from about 40 mg/kg to about 50 mg/kg biotin, from about 35 mg/kg to about 40 mg/kg biotin, from about 40 mg/kg to about 45 mg/kg biotin, or from about 45 mg/kg to about 50 mg/kg biotin, or from about 50 mg/kg to about 55 mg/kg biotin) can be administered to a non-human animal. For example, when a non-human animal described herein is a mouse, the mouse can be administered about 1 mg biotin.

In some cases, a polypeptide having ligase activity (e.g., biotin ligase activity) and having an ER retention signal can include a detectable label. In some cases, a detectable label can be a peptide tag. Examples of detectable labels that be included in a polypeptide having ligase activity and having an ER retention signal include, without limitation, an HA tag, a Myc-tag, a FLAG-tag, a T7-tag, and a V5-tag. For example, a nucleic acid (e.g., a transgene) that encodes a polypeptide having ligase activity (e.g., biotin ligase activity) and having an ER retention signal such that the polypeptide-encoding sequence is not operably linked to a promoter sequence, and instead includes an intervening nucleic acid sequence (e.g., a sequence including one or more stop codons) can include nucleic acid encoding an HA tag.

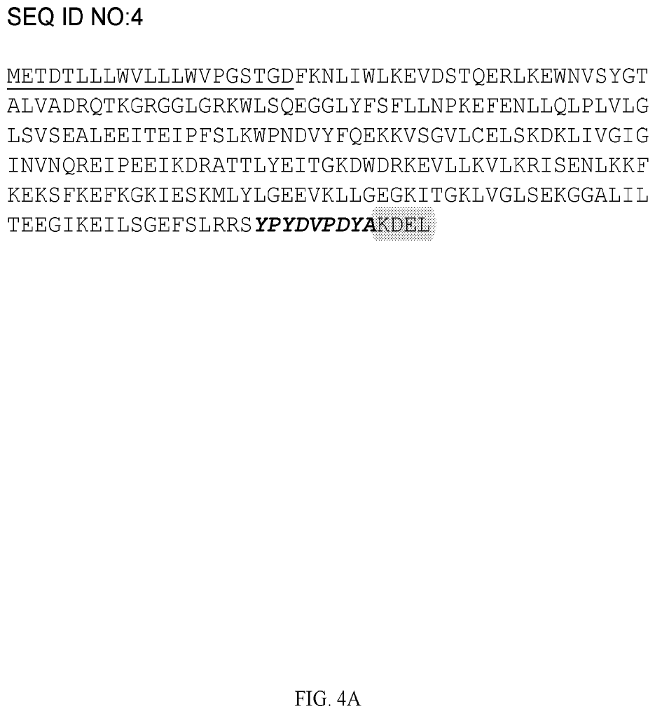

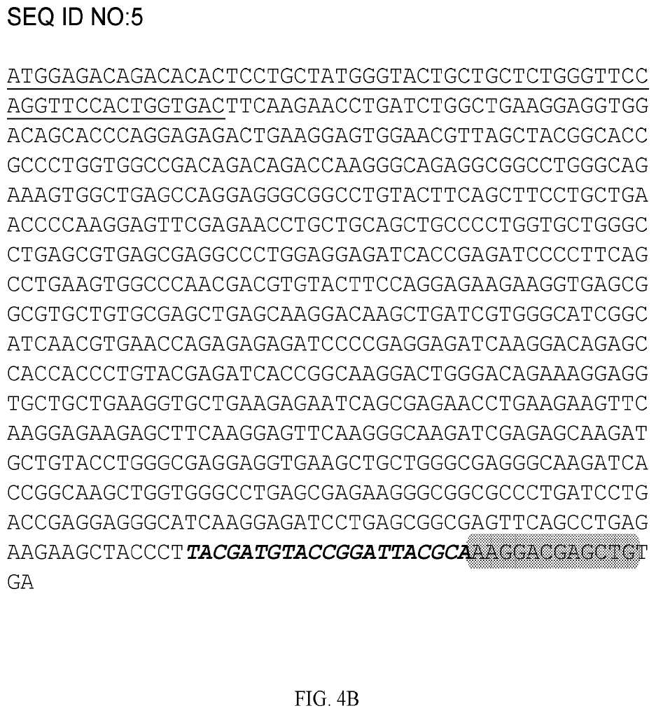

In some cases, a polypeptide having ligase activity (e.g., biotin ligase activity) and having an ER retention signal can include an ER retention signal, a biotin ligase, and an HA tag. For example, polypeptide having ligase activity and having an ER retention signal that includes an ER retention signal, a biotin ligase, and an HA tag can include the amino acid sequence set forth in FIG. 4A. In some cases, a nucleic acid (e.g., a transgene) that encodes a polypeptide having biotin ligase activity, having an ER retention signal, and having an HA tag can be as set forth in FIG. 4B.

Nucleic acid that encodes a polypeptide having ligase activity (e.g., biotin ligase activity) and having an ER retention signal can include a promoter sequence. A promoter sequence can be a naturally occurring promoter sequence or a recombinant promoter sequence. In some cases, a promoter sequence can be a synthetic promoter sequence. In some cases, a promoter sequence can be a constitutively active promoter sequence. In some cases, a promoter sequence can be a regulated (e.g., an inducible) promoter sequence. In some cases, a promoter sequence can be a tissue specific promoter sequence (e.g., a muscle-specific promoter or an endothelial-specific promoter). Examples of promoter sequences that can be included in a nucleic acid sequence encoding a polypeptide that includes an ER retention signal and ligase activity include, without limitation, a CAG promoter sequence (e.g., a P_{C}AG promoter sequence), a SV40 promoter sequence, a CMV promoter sequence, a UBC promoter sequence, a EF1A promoter sequence, and a PGK promoter sequence.

In some cases, nucleic acid that encodes a polypeptide having ligase activity (e.g., biotin ligase activity), having an ER retention signal, and having a promoter sequence can include an intervening nucleic acid sequence (e.g., a sequence including a stop codon) such that the polypeptide-encoding sequence is not operably linked to the promoter sequence. Any appropriate intervening sequence can be used. For example, an intervening sequence can include a stop codon that is in frame with the promoter sequence such that the nucleic acid that encodes a polypeptide having ligase activity and having an ER retention signal is not expressed.

In some cases, an intervening sequence (e.g., a sequence including a stop codon) can include a nucleic acid sequence encoding a marker polypeptide. For example, an intervening sequence can include a nucleic acid sequence encoding a marker polypeptide followed by a stop codon. Examples of marker polypeptides include, without limitation, a fluorescent polypeptide (e.g., a green fluorescent protein (GFP), a yellow fluorescent protein (YFP), and a cyan fluorescent protein (CFP)), luciferase, and LacZ beta-galactosidase. In some cases, a nucleic acid sequence encoding a marker polypeptide can include nucleic acid encoding a poly A terminator (e.g., a poly A tail).

When a nucleic acid described herein (e.g., a transgene) includes a promoter sequence, a first recombination site, an intervening nucleic acid sequence including nucleic acid encoding a marker polypeptide and one or more stop codons, a second recombination site, and nucleic acid encoding a polypeptide having ligase activity and having an ER retention signal, the nucleic acid sequence encoding a marker polypeptide can be operably linked to the promoter sequence. For example, an intervening sequence can include a nucleic acid sequence encoding a marker polypeptide that is operably linked to a promoter sequence followed by one or more stop codons that is in frame with the promoter sequence such that the nucleic acid sequence encoding the marker polypeptide is expressed, and the nucleic acid encoding the polypeptide having ligase activity and having the ER retention signal is not expressed.

When a nucleic acid described herein (e.g., a transgene) includes a promoter sequence and nucleic acid encoding a polypeptide having ligase activity and having an ER retention signal, the nucleic acid encoding the polypeptide having ligase activity and having the ER retention signal can be operably linked to the promoter sequence. In some cases, an intervening sequence including one or more stop codons (e.g., an intervening nucleic acid sequence including nucleic acid encoding a marker polypeptide and one or more stop codons) can be removed (e.g., by cre-lox recombination) from a nucleic acid described herein that includes a promoter sequence, a first recombination site, an intervening nucleic acid sequence including one or more stop codons, a second recombination site, and nucleic acid encoding a polypeptide having ligase activity and having an ER retention signal such that the nucleic acid encoding the polypeptide having ligase activity and having the ER retention signal is operably linked to the promoter sequence. For example, when an intervening sequence is removed, the promoter sequence can be operably linked to the nucleic acid encoding the polypeptide having ligase activity and having the ER retention signal, such that tissues or cell types in which the intervening sequence has been removed express polypeptides having ligase activity and having an ER retention signal.

The term “operably linked” as used herein refers to positioning a regulatory element (e.g., a promoter sequence) relative to a nucleic acid sequence encoding a polypeptide in such a way as to permit or facilitate expression of the encoded polypeptide. In some aspects of a nucleic acid described herein, for example, a promoter sequence can be positioned 5′ relative to a nucleic acid including a first recombination site, an intervening nucleic acid sequence including nucleic acid encoding an optional marker polypeptide and one or more stop codons, a second recombination site, and nucleic acid encoding a polypeptide having ligase activity and having an ER retention signal, and can facilitate expression of the encoded marker polypeptide. For example, a promoter sequence (e.g., a P_{CAG} promoter sequence) can be operably linked to nucleic acid including a first recombination site, an intervening nucleic acid sequence including nucleic acid encoding an optional marker polypeptide and one or more stop codons, a second recombination site, and nucleic acid encoding a polypeptide having ligase activity and having an ER retention signal, and can facilitate expression of the encoded marker polypeptide, but does not facilitate expression of the nucleic acid encoding the polypeptide having ligase activity and having the ER retention signal. In some aspects of a nucleic acid described herein, for example, a promoter sequence can be positioned 5′ relative to a nucleic acid including nucleic acid that encodes a polypeptide having ligase activity and having an ER retention signal (e.g., nucleic acid from which an intervening nucleic acid sequence including one or more stop codons has been removed via recombination), and can facilitate expression of the encoded polypeptide. For example, a promoter sequence (e.g., P_{CAG} promoter sequence) can be operably linked to a nucleic acid encoding a polypeptide including ligase activity and an ER retention signal and can facilitate expression of the encoded polypeptide having ligase activity and having an ER retention signal.

An intervening sequence can be removed using one or more recombinase polypeptides (e.g., a cre recombinase). In the presence of a recombinase polypeptide (e.g., a cre recombinase), the recombinase polypeptide can act on the recombinase recognition sites flanking the intervening sequence to induce recombination and excise the intervening sequence. For example, a transgenic mouse line that contains a transgene that includes a promoter sequence, followed by a first loxP site, followed by intervening nucleic acid sequence that includes one or more stop codons, followed by a second lox P site, followed by a nucleic acid sequence encoding a polypeptide having ligase activity and an ER retention signal can be crossed with a transgenic mouse line that expresses a cre recombinase. The offspring of such a cross can express the encoded polypeptide (e.g., the polypeptide including ligase activity and an ER retention signal) in selected cell types or tissues.

In some cases, an intervening sequence can be removed in a tissue-specific or cell-specific manner. For example, a transgenic mouse line that contains a transgene that includes a promoter sequence, followed by a first loxP site, followed by intervening nucleic acid sequence that includes one or more stop codons, followed by a second lox P site, followed by a nucleic acid sequence encoding a polypeptide including ligase activity and an ER retention signal can be crossed with a transgenic mouse line that expresses a cre recombinase in a tissue-specific or cell-specific manner. The offspring of such a cross can have those tissues or cell types that express the cre recombinase also expressing the encoded polypeptide (e.g., the polypeptide including ligase activity and an ER retention signal) with the tissues or cell types that do not express the cre recombinase also not expressing the encoded polypeptide.

In some cases, an intervening sequence can be removed in an endothelial-specific manner. For example, a transgenic mouse line that contains a transgene that includes a promoter sequence, followed by a first loxP site, followed by intervening nucleic acid sequence that includes one or more stop codons, followed by a second lox P site, followed by a nucleic acid sequence encoding a polypeptide including ligase activity and an ER retention signal can be crossed with a transgenic mouse line that expresses a cre recombinase in endothelial cells. A transgenic mouse that expresses a cre recombinase in endothelial cells can include in its genome an endothelial-specific promoter operably linked to a nucleic acid sequence encoding a Cre recombinase polypeptide. Examples of endothelial-specific promoters include, without limitation, a VE-cadherin promoter, an endoglin promoter, a Flt-1 promoter, an ICAM-2 promoter, a Tie-2-promoter, and an Apin-promoter. The offspring of such a cross can express the polypeptide including ligase activity and an ER retention signal in endothelial cells (or tissues containing endothelial cells).

In some cases, an intervening sequence can be removed in a muscle-specific manner. For example, a transgenic mouse line that contains a transgene that includes a promoter sequence, followed by a first loxP site, followed by intervening nucleic acid sequence that includes one or more stop codons, followed by a second lox P site, followed by a nucleic acid sequence encoding a polypeptide including ligase activity and an ER retention signal can be crossed with a transgenic mouse line that expresses a cre recombinase in myocytes. A transgenic mouse that expresses a cre recombinase in myocytes can include in its genome a muscle-specific promoter operably linked to a nucleic acid sequence encoding a Cre recombinase polypeptide. Examples of muscle-specific promoters include, without limitation, a MCK promoter, a desmin promoter, a Mb promoter, human skeletal actin promoter, and a Pax? promoter. The offspring of such a cross can express the polypeptide including ligase activity and an ER retention signal in myocytes (or in tissues containing myocytes).

Examples of tissue-specific or cell-specific cre recombinase transgenic mice that can be used as described herein include, without limitation, those set for in Table 1.

TABLE 1
Exemplary tissue-specific or cell-specific cre recombinase transgenic mice.

| Mouse strain name | promoter | Tissue/Cell site of Cre expression |
|---|---|---|
| B6.Cg-Tg(ACTA1-cre)79Jme/J | ACTA1 | Muscle cells of somites and heart |
| B6. Cg-Tg(Alb-cre)21Mgn/J | Alb | Liver |
| B6.Cg-Tg(Cdh5-cre)7Mlia/J | Cdh5 | Endothelium |
| B6.FVB(129S4)-Tg(Ckmm-cre)5Khn/J | Ckmm | Skeletal and cardiac muscle |
| B6.Cg-Tg(Fabp4-cre)1Rev/J | Fabp | Brown and white adipose tissue |
| B6;129S4-Foxd1^{tm1(GFP/cre)Amc}/J | Foxd1 | Kidney |
| B6.Cg-Tg(Ins2-cre)25Mgn/J | Ins2 | Pancreatic beta cells |
| STOCK Tg(KRT14-cre)1Amc/J | KRT14 | Skin |
| B6.Cg-Tg(Lck-cre)548Jxm/J | Lck | Thymocytes |
| B6.129-Lepr^{tm2(cre)Rck}/J | Lepr | Hypothalamus |
| B6;129P2-^{Lyve1tm1.1(EGFP/cre)Cys}/J | Lyve1 | Lymphatic endothelium |
| B6.129S1-Mnx1^{tm4(cre)Tmj}/J | Mnx1 | Motor neurons |
| B6.129S4-Myf5^{tm3(cre)Sor}/J | Myf5 | Skeletal muscle and the dermis |
| B6.FVB(129)-A1cf^{Tg(Myh6-cre/Esr1}*^{)1Jmk}/J | Myh6 | Cardiac myocytes |
| B6.FVB-Tg(Myh6-cre)2182Mds/J | Myh6 | Cardiac myocytes |
| B6.Cg-Tg(Nes-cre)1Kln/J | Nes | Central and peripheral nervous system |
| B6.129S6-Tagln^{tm2(cre)Yec}/J | Tagln | Smooth muscle cells |
| B6.Cg-Tg(Tek-cre)1Ywa | Tek | Endothelial cells |
| B6.Cg-Tg(Tek-cre)12Flv/J | Tek | Endothelial cells |

Additional examples of tissue-specific or cell-specific cre recombinase transgenic mice that can be used as described herein include, without limitation, those set for in jax.org/research-and-faculty/resources/cre-repository.

This document also provides methods and materials for using transgenic non-human animals described herein (e.g., transgenic non-human animals having tissue-specific secretion of tagged molecules). In some cases, non-human animals described herein can be used to identify molecules (e.g., polypeptides) secreted by a particular cell type (or a tissue containing that particular cell type). For example, when a transgenic non-human animal described herein secretes tagged molecules from endothelial cells, the transgenic non-human animal can be used to identify tagged molecules (e.g., tagged polypeptides) secreted by endothelial cells (or a tissue containing endothelial cells). For example, when a transgenic non-human animal described herein secretes tagged molecules from myocytes, the transgenic non-human animal can be used to identify tagged molecules (e.g., tagged polypeptides) secreted by myocytes (or a tissue containing myocytes).

In some cases, non-human animals described herein (e.g., transgenic non-human animals having tissue-specific secretion of tagged molecules) can be used to identify molecules (e.g., polypeptides) secreted by a particular cell type (or a tissue containing that particular cell type) under stress. In some cases, a stress can be a physiological stress. Examples of stresses can include, without limitation, exercise, fasting, over-feeding, changes in external temperature, psychological stress, and pain. For example, a transgenic non-human animal described herein can be subjected to a stress and can be used to identify molecules (e.g., polypeptides) secreted by a particular cell type (or a tissue containing that particular cell type) in the presence of the stress. In some cases, a transgenic mouse having cardiac-specific secretion of tagged molecules can be subjected to a stress such as exercise and can be used to identify molecules secreted by cardiomyocytes (or a tissue containing cardiomyocytes) in the presence of stress.

In some cases, non-human animals described herein (e.g., transgenic non-human animals having tissue-specific secretion of tagged molecules) can be used to identify molecules (e.g., polypeptides) secreted by a particular cell type (or a tissue containing that particular cell type) associated with a disease (e.g., the presence of a disease and/or progression of a disease). Examples of diseases for which non-human animals described herein can be used to identify molecules (e.g., polypeptides) secreted by a particular cell type (or a tissue containing that particular cell type) include, without limitation, cancer, autoimmune conditions, heart diseases, neurological disorders (e.g., Alzheimer's disease), endocrine disorders (e.g., diabetes), kidney diseases (e.g., polycystic kidney disease), pancreatic diseases (e.g., pancreatitis), and stroke.

In some cases, a transgenic non-human animal having tissue-specific secretion of tagged molecules can be crossed with a non-human animal having or being likely to develop a particular disease (e.g., a non-human animal having or likely to develop a particular disease that is of the opposite sex and of the same species as the transgenic non-human animal having tissue-specific secretion of tagged molecules), and the progeny having tissue-specific secretion of tagged molecules and having or being likely to develop the disease can be used to identify molecules secreted by a particular cell type (or a tissue containing that particular cell type) associated with a disease. For example, a transgenic mouse having tissue-specific secretion of tagged molecules (e.g., polypeptides) can be crossed with (e.g., mated with) a mouse model for a disease, and progeny having tissue-specific secretion of tagged molecules (e.g., polypeptides) and having or being likely to develop the disease can be used to identify molecules secreted by cells (or a tissue containing the cells) in the disease (e.g., can be used to identify circulating biomarkers of the disease). Examples of mouse model for diseases can include, without limitation, a mouse model for Alzheimer's disease, a diabetic mouse model, and a mouse model for polycystic kidney disease. For example, a transgenic mouse having neuron-specific secretion of tagged molecules (e.g., polypeptides) can be crossed with (e.g., mated with) a mouse model for Alzheimer's disease, and progeny having neuron-specific secretion of tagged molecules (e.g., polypeptides) and having or being likely to develop Alzheimer's disease can be used to identify molecules secreted by neurons (or a tissue containing neurons) in Alzheimer's disease (e.g., can be used to identify circulating biomarkers of Alzheimer's disease). For example, a transgenic mouse having adipocyte-specific secretion of tagged molecules (e.g., polypeptides) can be crossed with (e.g., mated with) a diabetic mouse model, and progeny having adipocyte-specific secretion of tagged molecules (e.g., polypeptides) and having or being likely to develop diabetes can be used to identify molecules secreted by adipocytes (or a tissue containing adipocytes) in diabetes (e.g., can be used to identify circulating biomarkers of diabetes). For example, a transgenic mouse having kidney-specific secretion of tagged molecules (e.g., polypeptides) can be crossed with (e.g., mated with) a mouse model for polycystic kidney disease, and progeny having kidney-specific secretion of tagged molecules (e.g., polypeptides) and having or being likely to develop polycystic kidney disease can be used to identify molecules secreted by kidney cells (or a tissue containing kidney cells) in polycystic kidney disease (e.g., can be used to identify circulating biomarkers of polycystic kidney disease).

In some cases, a transgenic non-human animal having tissue-specific secretion of tagged molecules can be crossed with a non-human animal having or being likely to develop cancer (e.g., a non-human animal having or likely to develop cancer that is of the opposite sex and of the same species as the transgenic non-human animal having tissue-specific secretion of tagged molecules), and the progeny having tissue-specific secretion of tagged molecules and having or being likely to develop the cancer can be used to identify molecules secreted by a particular cell type (or a tissue containing that particular cell type) associated with the cancer. For example, a transgenic mouse having cell-specific secretion of tagged molecules (e.g., polypeptides) can be crossed with (e.g., mated with) a mouse having or being likely to develop cancer, and progeny having cell-specific secretion of tagged molecules and having or being likely to develop cancer can be used to identify molecules (e.g., polypeptides) secreted by the cancer cells (e.g., can be used to identify circulating biomarkers of the cancer). For example, a transgenic mouse having pancreatic-specific secretion of tagged molecules can be crossed with (e.g., mated with) a mouse model for pancreatic cancer, and progeny having pancreatic-specific secretion of tagged molecules (e.g., polypeptides) and having or being likely to develop pancreatic cancer can be used to identify molecules (e.g., polypeptides) secreted by pancreatic cancer cells (or a tissue containing pancreatic cancer cells). For example, a transgenic mouse having osteocyte-specific secretion of tagged molecules can be crossed with (e.g., mated with) a mouse model for bone cancer, and progeny having osteocyte-specific secretion of tagged molecules (e.g., polypeptides) and having or being likely to develop bone cancer can be used to identify molecules (e.g., polypeptides) secreted by bone cancer cells (or a tissue containing bone cancer cells). For example, a transgenic mouse having kidney cell-specific secretion of tagged molecules (e.g., polypeptides) can be crossed with (e.g., mated with) a mouse model for kidney cancer, and progeny having kidney cell-specific secretion of tagged molecules (e.g., polypeptides) and having or being likely to develop kidney cancer can be used to identify molecules secreted by kidney cancer cells (or a tissue containing kidney cancer cells).

In some cases, a disease can be induced in a transgenic non-human animal having tissue-specific secretion of tagged molecules, and the transgenic non-human animal having tissue-specific secretion of tagged molecules and having the disease can be used to identify molecules secreted by a particular cell type (or a tissue containing that particular cell type) associated with the disease. For example, a disease can be induced in a transgenic mouse having tissue-specific secretion of tagged molecules (e.g., polypeptides), and the transgenic mouse having tissue-specific secretion of tagged molecules (e.g., polypeptides) and having the disease can be used to identify molecules secreted by cells (or a tissue containing the cells) in the disease (e.g., can be used to identify circulating biomarkers of the disease). Examples of diseases that can be induced in a transgenic mouse having tissue-specific secretion of tagged molecules described herein can include, without limitation, heart failure, pancreatitis, stroke, and diabetes. For example, a surgery to band the aorta thereby causing cardiac hypertrophy can be performed on a transgenic mouse having cardiac-specific secretion of tagged molecules (e.g., polypeptides) to induce heart failure in the mouse, and the transgenic mouse model having cardiac-specific secretion of tagged molecules (e.g., polypeptides) and having heart failure can be used to identify molecules secreted by cardiomyocytes (or a tissue containing cardiomyocytes) during heart failure (e.g., can be used to identify circulating biomarkers of heart failure). In another example, cerulein can be administered to a transgenic mouse having pancreatic-specific secretion of tagged molecules (e.g., polypeptides) to induce pancreatitis in the mouse, and the transgenic mouse model having pancreatic-specific secretion of tagged molecules (e.g., polypeptides) and having pancreatitis can be used to identify molecules secreted by pancreatic cells (or a tissue containing pancreatic cells) during pancreatitis (e.g., can be used to identify circulating biomarkers of pancreatitis). In another example, a surgery to ligate an artery can be performed on a transgenic mouse having endothelial-specific secretion of tagged molecules (e.g., polypeptides) to induce stroke in the mouse, and the transgenic mouse model having endothelial-specific secretion of tagged molecules (e.g., polypeptides) and having stroke can be used to identify molecules secreted by endothelial cells (or a tissue containing endothelial cells) during stroke (e.g., can be used to identify circulating biomarkers of stroke). In another example, sterptazotocin can be administered to a transgenic mouse having adipocyte-specific secretion of tagged molecules (e.g., polypeptides) to induce diabetes in the mouse, and the transgenic mouse model having adipocyte-specific secretion of tagged molecules (e.g., polypeptides) and having diabetes can be used to identify molecules secreted by adipocytes (or a tissue containing adipocytes) in diabetes (e.g., can be used to identify circulating biomarkers of diabetes).

Any appropriate method can be used to identify tagged molecules (e.g., polypeptides) secreted by a non-human animal described herein (e.g., a transgenic non-human animal having tissue-specific secretion of tagged molecules). In some cases, polypeptide isolation and sequencing techniques can be used to identify tagged polypeptides secreted by a transgenic non-human animal having tissue-specific secretion of tagged polypeptides.

In some cases, one or more tagged molecules (e.g., polypeptides) secreted by a non-human animal described herein (e.g., a transgenic non-human animal having tissue-specific secretion of tagged molecules) can be isolated from a sample obtained from the non-human animal. A sample can be any appropriate sample. In some cases, a sample can be a biological sample. In some cases, a sample can contain one or more biological molecules (e.g., nucleic acids such as DNA and RNA, polypeptides, carbohydrates, lipids, hormones, and/or metabolites). Examples of samples from which one or more tagged molecules (e.g., polypeptides) secreted by a non-human animal described herein can be isolated from include, without limitation, tissue samples (e.g., tumor tissues such as those obtained by biopsy, explanted tissue, and explanted organs), fluid samples (e.g., whole blood, serum, plasma, urine,CSF, and saliva), and cellular samples (e.g., buccal samples). A sample can be a fresh sample or a fixed sample (e.g., a formaldehyde-fixed sample or a formalin-fixed sample). In some cases, a sample can be a processed sample (e.g., an embedded sample such as a paraffin or OCT embedded sample). Any appropriate method can be used to isolate one or more tagged molecules (e.g., polypeptides) secreted by a non-human animal described herein (e.g., a transgenic non-human animal having tissue-specific secretion of tagged molecules). In some cases, affinity purification (e.g., affinity purification selected based on the tag) can be used to isolate one or more tagged molecules. For example, when a tagged molecule is a biotinylated molecule, the tagged molecule can be isolated using streptavidin purification and/or avidin purification. Once a tagged molecule has been isolated, the tagged molecule can be identified. Any appropriate can be used to identify a tagged molecule (e.g., polypeptides) secreted by a transgenic non-human animal having tissue-specific secretion of tagged molecules. For example, mass spectroscopy, antibody array, ELISA, and/or Western blotting can be used to identify a tagged polypeptide.

The invention will be further described in the following examples, which do not limit the scope of the invention described in the claims.

### EXAMPLES

### Example 1: A Genetic Platform to Delineate In Vivo Tissue-Specific Secretion

This example describes using proximity biotinylation (see, e.g., Kim et al., Trends Cell Biol.; 26:804-817 (2016); and Kim et al., Proc Natl Acad Sci U S A;111:E2453-61 (2014)) to identify and/or capture a tissue-specific secretome. In this example, a proximity-dependent biotin identification (BioID) construct encoding a bacterial enzyme called BirA was fused in-frame to a human influenza hemagglutinin (HA)-epitope tag and an endoplasmic reticulum (ER) retention sequence (e.g., a KDEL peptide C-terminal ER retention sequence) to create a construct encoding an ER-BioID^{HA} polypeptide having the ability to constitutively localize to the lumen of the ER and to biotinylate polypeptides that transit through the ER. For example, a cell's membrane and secreted polypeptides that must transit through the ER can be biotinylated by the ER localized ER-BioID^{HA} polypeptides.

### BioID2-ER Construct

To construct an ER lumen-resident BioID (termed BioID2-ER) expression plasmid, a coding sequence was designed with the following features in order: an IgK signal peptide, a BioID2 coding sequence, an HA tag, and an ER retention signal KDEL (Lys-Asp-Glu-Leu; SEQ ID NO:1) tetra peptide. In brief, the primers listed in Table 2 were used to amplify BioID2-ER using a plasmid MCS-13XLinker-BioID2-HA (Biofront Technologies Addgene #80899) as a template. The resulting DNA fragment was cloned into Nhe/BsrGI sites of lentiviral vector pLJM1-EGFP (Addgene #19319), resulting in pLJM1-ER-BioID, and was confirmed by DNA sequencing.

TABLE 2
Primers used for PCR genotyping and cloning.

| | | | SEQ |
|---|---|---|---|
| | Primer | | ID |
| Use | name | Primer sequence | NO |
| BioID2-ER | BioID-F | CTAGAGCCTCTGCTAACCATG | 9 |
| genotyping | | | |
| | BioID- | CAGATCAGGTTCTTGAAGTCA | 10 |
| | R | CCAGT | |
| Mck-Cre | MckCre- | TAAGTCTGAACCCGGTCTGC | 11 |
| | F | | |
| genotyping | McKCre- | GTGAAACAGCATTGCTGTCA | 12 |
| | R | CTT | |
| VE- | CdhCre- | GCGGTCTGGCAGTAAAAACTA | 13 |
| Cadherin- | F | TC | |
| Cre | | | |
| genotyping | | | |
| | CdhCre- | GTGAAACAGCATTGCTGTCACT | 14 |
| | R | T | |
| BioID2 | Forward | tccgctogcATGGAGACAGACA | 15 |
| cloning | | CACTCCTGCTATGGGTACTGCT | |
| | | GCTCTGGGTTCCAGGTTCCACT | |
| | | GGTGACTTCAAGAACCTGATCT | |
| | | GGCTGAAGGAGG | |
| | | (IgK signal peptide | |
| | | coding sequence | |
| | | is in bold) | |
| | Reverse | ACTTGTACACACAGCTCGTCC | 16 |
| | | CTTTGCGTAATCGGTACATCG | |
| | | TAAGGGTAGC | |
| | | (KDEL (SEQ ID NO: 1) | |
| | | coding sequence | |
| | | in bold) | |

### Cell Culture and Transfection

Human umbilical vein endothelial cells (HUVECs) (Lonza, Cat. No. C2519A) were grown in the endothelial cells growth medium (PromoCell, Cat. No. C22010). To express BioID2-ER in HUVECs, the cells were infected with BioID2-ER lentiviruses as described elsewhere (see, e.g., Wang et al., “Viral Packaging and Cell Culture for CRISPR-Based Screens,” Cold Spring Harb Protoc 2016, doi:10.1101/pdb.prot090811 (2016)). For induction of an EndoMT, sub-confluent HUVECs grown on fibronectin-coated plates were incubated with 10 ng/mL TGF-β1 (PeproTech, Cat. No. 100-21C) and 1 ng/mL IL-1β (PeproTech, Cat. No. 200-01B) for 3 or 4 days. For protein biotinylation, HUVECs were incubated with 50 μM biotin for 6 hours. After washing, the cells were further cultured overnight. The conditioned medium was then collected and incubated with Streptavidin beads (Invitrogen) overnight. Beads were collected using a magnetic stand and washed as described elsewhere (see, e.g., Kim et al., Mol Biol Cell 27:1188-1196 (2016)). Endothelial cell lysates were prepared in RIPA buffer, analyzed by Western blotting using antibodies against Streptavidin, Alexa Fluor™ 488, P4HA2 antibody (PAS-27761) (Invitrogen), anti-Pentraxin 3/PTX3 (EPR6699) (Abcam), anti-Laminin-5, clone D4B5 (EMD Millipore), anti-α Tubulin (B-7), or anti-β-Actin (C4), (Santa Cruz Biotechnology).

### Generation of Tissue-Specific BioID-ER Transgenic Mice

The ER-BioID^{HA} coding fragment was cloned into EcoRI/NheI sites of pCLE vector, resulting in a transgenic vector pCLE-ER-BioID^{HA} in which the ER-BioID^{HA} coding sequence was under the control of the CAG promoter (P_{CAG}, a combination of the cytomegalovirus early enhancer element and chicken β-actin promoter). A loxP-Stop-loxP cassette (LSL; a DNA fragment containing EGFP coding sequence followed by a poly A terminator) was placed between P_{CAG} and BioID2-ER (see FIG. 2A). After removing the non-relevant part by Xhol and DraIII digestions, the transgenic vector was microinjected into the pronuclei of fertilized eggs to generate transgenic mice (C57BL/6 background). The founder lines were genotyped by PCR analyses using the primers listed in Table 2. The transgenic mice do not express the ER-BioID^{HA} transgene until bred with cell-type specific Cre-transgenic mice to remove the LSL cassette and place P_{CAG} adjacent to the ER-BioID^{HA} coding sequence (FIG. 2A). Thus, endothelial-specific expressing mice (BioID-ER^{EC}) were obtained by breeding the LSL-BioID2-ER mice with Cdh5-Cre (Cre-recombinase under the VE-cadherin promoter) mice (The Jackson Laboratory #006137) to delete the LSL cassette only in ECs. Muscle-specific BioID-ER-expressing mice BioID-ER^{SK} were generated similarly using the corresponding muscle specific MCK-Cre mice (The Jackson Laboratory #006475). ER-BioID^{HA} transgenic mice were generated by the University of Pittsburgh Transgenic and Gene Targeting Laboratory.

### Animal Studies

For biotin labelling of secretome, 24 hours prior to serum collection, the mice were administered both intraperitoneally (I.P.) and subcutaneously with 500 μL biotin (2 mg/mL) (Sigma), and fed on diet soaked in biotin solution. The sera were collected after terminally bleeding. 300-μL serum from each mouse was incubated with 100-μL streptavidin beads overnight. The beads were collected using a magnetic stand, and the biotinylated proteins were collected in SDS loading buffer and subjected for mass spectrometry. Unless stated otherwise, peptide counts represent the pooled serum from two or more mice. For exercise, mice were provided with InnoWheel on top of an InnoDome for a month. In each cage, there was one apparatus for every two mice.

### Mass Spectrometry

Each sample was heated at 85° C. for 5 minutes and separated on a 10% Bis-Tris Novex mini-gel (Invitrogen) using the MES buffer system. The gel was stained with coomassie, and each lane was excised into ten equally sized segments. Gel segments were reduced using dithiothreitol, alkylated with iodoacetamide, and then subjected to digestion with trypsin (Promega, Madison, Wis.). Digests were analyzed by nano LC/MS/MS with a NanoAcquity HPLC system (Waters, Milford, Mass.) interfaced to a Fusion Lumos tandem mass spectrometer (ThermoFisher, San Jose, Calif.). Peptides were loaded on a trapping column and eluted over a 75 μm analytical column at 350 nL/minute; both columns were packed with Luna C18 resin (Phenomenex, Torrance, Calif.). A 30 minute gradient was employed for each segment. The mass spectrometer was operated in data-dependent mode, with MS and MS/MS performed in the Orbitrap at 60,000 FWHM resolution and 15,000 FWHM resolution, respectively. APD was turned on. The instrument was run with a 3 second cycle for MS and MS/MS. Dynamic exclusion and repeat settings ensured each ion was selected only once and excluded for 30 seconds thereafter. Product ion data were searched against the combined forward and reverse Swissprot H. sapiens protein database using a locally stored copy of the Mascot search engine v2.6 (Matrix Science, London, U.K.) via Mascot Daemon v2.6. Peak lists were generated using the Proteome Discoverer v2.1 (ThermoFisher). The database was appended with common background proteins. Search parameters were precursor mass tolerance 10 ppm, product ion mass tolerance 0.02 Da, 2 missed cleavages allowed, fully tryptic peptides only, fixed modification of carbamidomethyl cysteine, variable modifications of oxidized methionine, protein N-terminal acetylation and pyro-glutamic acid on N-terminal glutamine. Mascot search result flat files (DAT) were parsed to the Scaffold software v4.8 (Proteome Software); data were filtered 1% protein and peptide level false discovery rate (FDR) and requiring at least two unique peptides per protein.

### Immunostaining

Endothelial cells were grown in tissue culture treated glass slides (BD Biosciences, 354104), washed with PBS, and fixed in PBS supplemented with 4% paraformaldehyde (Electron Microscopy Sciences) for 10 minutes at room temperature. After permeabilization with 0.3% Triton X-100 (Sigma-Aldrich) in PBS for 20 minutes at room temperature, the cells were washed with PBS and blocked in Fluorescent Blocking Buffer (Thermo Fisher Scientific) for 1 hour at room temperature. Cells were then probed with mouse antibody against HA-Tag (sc-7392) (Santa Cruz Biotechnology) and rabbit antibody against Calnexin (C5C9) (Cell Signaling) overnight at 4° C., followed by labeling with a goat anti-mouse Texas Red and Alexa Fluor-488 donkey anti-rabbit antibodies (Thermo Fisher Scientific).

### Statistical Analyses

GraphPad Prism was used for statistical analyses. The two-tailed unpaired Student's t test was used. P<0.05 was considered as a significant difference. In comparisons between two groups with equal variance, unpaired two-tailed Student's t-tests was used to identify significant (P<0.05) differences.

### Results

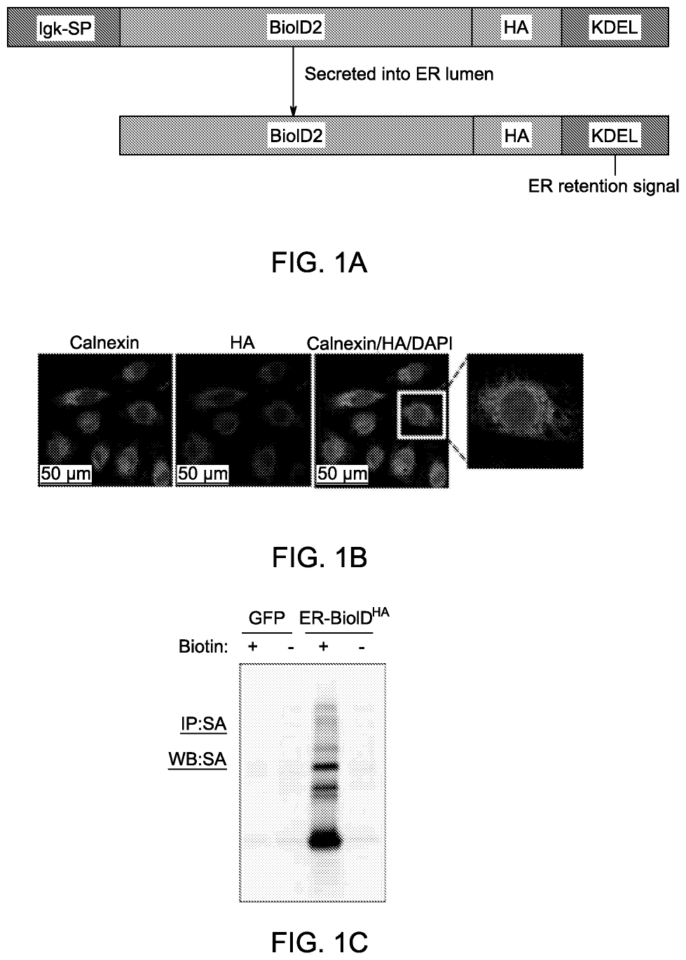

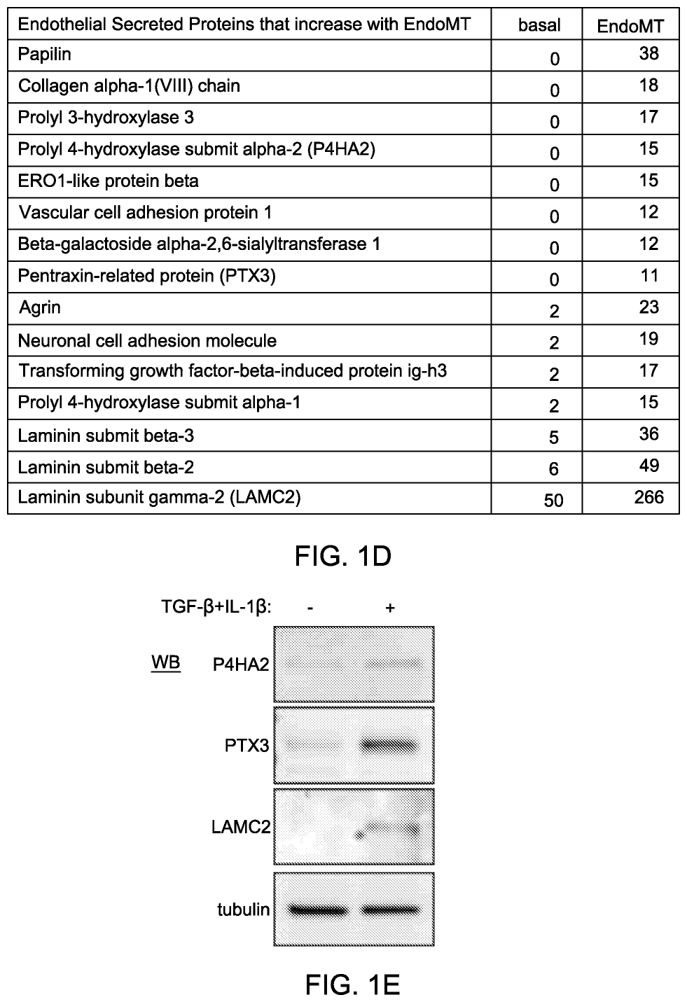

To tag secreted polypeptides ER-BioID^{HA} was constructed to contain the promiscuous biotinylation enzyme BioID2, in-frame with an HA-epitope tag and the KDEL (SEQ ID NO:1) peptide C-terminal ER retention sequence (SEQ ID NO:1; FIG. 1A). This construct was packaged in a lentivirus in order to allow for expression in a wide variety of cell types. When expressed in endothelial cells, ER-BioID^{HA} extensively co-localized with the ER resident protein calnexin (FIG. 1B). The supernatants of primary endothelial cells infected with a lentivirus expressing either ER-BioID^{HA} or a GFP control in the presence or absence of exogenous biotin were analyzed. Supernatants were affinity purified with streptavidin beads and analyzed by streptavidin-based Western blotting. In GFP-expressing endothelial cells, few streptavidin purified proteins were present in the supernatant either in the presence or absence of exogenously added biotin (FIG. 1C). In contrast, in the presence of exogenous biotin (50 μM), endothelial cells expressing ER-BioID^{HA} generated multiple biotinylated proteins that could be recovered in the conditioned medium (FIG. 1C).

Endothelial cells stimulated with a cytokine cocktail of TGF-β1 and IL-1β undergo conversion from an endothelial to mesenchymal phenotype (EndoMT), and this cell-based model was used to see if ER-BioID^{HA} expression provides a rapid way of characterizing the secretome of cells undergoing phenotypic conversion. The conditioned media of ER-BioID^{HA} expressing cells was analyzed under basal conditions and three days after being stimulated with TGF-β1 and IL-1β. Streptavidin purification of the conditioned medium from endothelial ER-BioID^{HA} expressing cells followed by mass spectroscopic identification provided a comprehensive compendium of proteins secreted under basal conditions and during EndoMT. Using this information, secreted proteins whose abundance increased (or decreased) following the induction of EndoMT were rapidly identified (FIG. 1D). Included in the list of proteins that increased during EndoMT were the extracellular matrix protein papilin (PAPLN), as well as collagen Type 8 and related proteins (COL8A1, P3H3, P4HA2). The increase in collagen-related proteins was consistent with the transition from an endothelial to mesenchymal phenotype. An increase in endothelial-specific membrane proteins such as VCAM-1, whose shedding into the media might be accelerated during the EndoMT process, was also observed. The overall fidelity of this approach was confirmed by analyzing lysates of uninfected endothelial cells undergoing EndoMT and analyzing some of the top ‘hits’ for which there were readily available antibodies. This analysis demonstrated that proteins found to increase in the media following EndoMT were in fact markedly induced in endothelial cells undergoing mesenchymal conversion (FIG. 1E). Thus, this strategy can be used to rapidly identify the endothelial secretome and more importantly, allows for the purification and identification of proteins that undergo conventional secretion and/or are shed from the membrane in a wide variety of cell types and conditions.

To examine if ER-BioID^{HA} expression also provides an in vivo method for detecting protein secretion, a transgenic mouse (termed the “Secretome Mouse”) was generated in which ER-BioID^{HA} expression was determined by excision of a floxed EGFP cassette (FIG. 2A). Crossing this line with a tissue specific Cre-recombinase allows for restricted, tissue-specific ER-BioID^{HA} expression. A mouse line for endothelial-specific expression of ER-BioID^{HA} was generated by crossing the transgenic secretome mouse line to mice expressing VE-cadherin Cre. In an effort to raise the serum concentrations of biotin to levels required for efficient proximity biotinylation, mouse chow was soaked in a biotin-containing solution. Serum from mice supplemented with biotin and expressing ER-BioID^{HA} with or without VE-cadherin Cre were purified by streptavidin and then analyzed by mass spectroscopy. From the complicated mix of serum proteins, streptavidin purification was able to identify a number of proteins that were observed exclusively, or with much higher abundance, in the serum of ER-BioID^{HA}/VE-cadherin Cre⁺ mice when compared to ER-BioID^{HA} expressing mice lacking Cre expression (FIG. 2B). Some polypeptides secreted from endothelial cells included well known endothelial cell surface receptors (MRC1, VEGFR-3, VCAM1, PECAM1, CDH5), von Willebrand factor (VWF), and coagulation factor VIII. The identification of these well-validated endothelial-specific proteins suggests that the Secretome Mouse represents a robust strategy for the deconvolution of serum to rapidly identify tissue-specific secretion. One polypeptide secreted from endothelial cells identified from the ER-BioID^{HA}/VE-cadherin Cre⁺ mice was fibrocystin (Pkhd111), a surface protein with a large extracellular domain.

In another example, the transgenic secretome mouse mice were crossed with mice expressing MCK-Cre. This led to ER-BioID^{HA} expression in skeletal muscle and cardiac muscle, but not in other tissues (FIG. 2C). Mice with MCK-Cre expression that did or did not have the ER-BioID^{HA} transgene were compared. Analysis of skeletal muscle cell lysate demonstrated that in the presence of biotin, ER-BioID^{HA}/MCK-Cre⁺ mice had multiple biotinylated proteins not evident in the control ER-BioID^{HA}/WT animals (FIG. 2D). Again, streptavidin purification of serum from ER-BioID^{HA}/MCK-Cre⁺ mice and the control ER-BioID^{HA}/WT animals followed by mass spectroscopy allowed for the identification of multiple proteins that derive from either skeletal muscle or heart. One polypeptide secreted from myocytes was myostatin, a known secreted muscle-derived factor, testifying to the fidelity and specificity of this platform.

To further demonstrate the potential utility of this system, ER-BioID^{HA}/MCK-Cre⁺ mice were separated into cages with or without a running wheel in order to determine how the muscle-derived secretome would change in the presence or absence of one month of voluntary exercise. Samples from these mice were subjected to mass spectroscopy, and data sets delineating proteins derived from either cardiac or skeletal muscle that increased or decreased with exercise were generated (FIG. 2E). While myostatin was one of the most abundant proteins detected under basal conditions, its abundance decreased with exercise (43 peptide counts basally vs. 27 peptide counts with exercise), with the percentage reduction in peptide counts closely mirroring the magnitude of reduction in circulating myostatin levels observed when human subjects exercise (see, e.g., Hittel et al., Med Sci Sports Exerc.; 42:2023-9 (2010)). Several of the muscle-derived proteins that appear to increase with exercise are of interest and may provide potential clues as to how exercise might confer systemic benefit.

In summary, these results demonstrate that ER-BioID^{HA} polypeptides in the ER lumen can biotinylate secreted polypeptides destined for conventional secretion, and can be used to identify the origin (e.g., the cellular origin and/or tissue origin) of secreted polypeptides. For example, the transgenic secretome mouse provided herein provides a genetic platform to identify the in vivo cell or tissue-specific secretome under basal conditions or following a physiological or pathophysiological stress. This model can be used as a discovery platform, as evident by the observations with exercise. This model also can be used to aid in the identification of circulating biomarkers such as disease biomarkers.

### Example 2: The Secretome Mouse Provides a Genetic Platform to Delineate Tissue-Specific In Vivo Secretion

This example describes the generation of a genetic model called the secretome mouse. The transgenic secretome mouse, which employs proximity biotinylation, provides a robust strategy for delineating the tissue-specific secretome as demonstrated here by identifying a set of in vivo proteins secreted by endothelial cells as well as by skeletal muscle with and without exercise. Some of the experiments described here are as described in the above analysis (Example 1) but were repeated using additional samples.

### Methods

### BioID2-ER Construct

To construct an ER lumen-resident BioID expression plasmid (termed ER-BioID^{HA}), the BioID coding sequence was designed with the following features, in order: IgK signal peptide, BioID2 coding sequence, HA tag, and the ER retention signal KDEL (Lys-Asp-Glu-Leu; SEQ ID NO:1) tetrapeptide. In brief, the primers listed in Table 2 were used to amplify BioID2-ER using a plasmid MCS-13XLinker-BioID2-HA (Biofront Technologies) as a template. The resulting DNA fragment was cloned into the Nhe/BsrGI sites of lentiviral vector pLJM1-EGFP (Addgene no. 19319), resulting in pLJM1-ERBioID^{HA}, which was confirmed by DNA sequencing.

### Human Umbilical Vein Endothelial Cell Culture, Transfection, and Expression

Human umbilical vein endothelial cells (HUVECs) (Lonza, Catalog No. C2519A) were grown in endothelial cells growth medium (PromoCell, Catalog No. C22010). To express ER-BioID^{HA} in HUVECs, the cells were infected overnight with ER-BioID^{HA} lentivirus (multiplicity of infection=10) or a control lentivirus consisting of either the pLJM1 empty vector or a similar GFP-expressing lentivirus. For induction of EndoMT, subconfluent HUVECs (ER-BioID^{HA}) grown on fibronectin-coated plates were incubated with 10-ng/mL TGF-β1 (PeproTech, Catalog No. 100-21C) and 1-ng/mL IL-1β (PeproTech, Catalog No. 200-01B) for 3 or 4 days. For protein biotinylation, HUVECs were pulsed with 50-μM biotin for 6 hours on day 2 or day 3. After washing to remove remaining biotin in the media, the cells were further cultured overnight. The conditioned medium was then collected and incubated with streptavidin beads (Invitrogen) overnight. Beads were collected using a magnetic stand and washed. Endothelial cell lysates were prepared in radioimmunoprecipitation assay buffer, analyzed by Western blotting using antibodies against streptavidin, Alexa Fluor 488 (Invitrogen, S32354), anti-pentraxin 3/PTX3 (Abcam, EPR6699), or anti-α tubulin (Santa Cruz Biotechnology, sc-5286). Protein levels of human pentraxin 3, transforming growth factor β (TGFβ)-induced protein IG-H3 (TGFβ IGH3), and soluble VCAM-1 in the above conditioned media were measured by ELISA using the respective commercial ELISA kits flowing the manufacturer manuals (R&D Systems, DPTX30B; Abcam, ab155426; and Invitrogen, BMS232).

### Immunostaining

HUVECs were grown on tissue culture treated glass slides (BD Biosciences, 354104), washed with phosphate-buffered saline (PBS), and fixed in PBS supplemented with 4% paraformaldehyde (Electron Microscopy Sciences) for 10 minutes at room temperature. After permeabilization with 0.3% Triton X-100 (Sigma-Aldrich) in PBS for 20 minutes at room temperature, the cells were washed with PBS and blocked in fluorescent blocking buffer (Thermo-Fisher Scientific, Catalog No. 37565) for 1 hour at room temperature. To verify the ER localization of ER-BioID^{HA}, cells were probed with a mouse antibody against the HA-Tag (Santa Cruz Biotechnology, sc-7392) and a rabbit antibody against calnexin (Cell Signaling, C5C9) overnight at 4° C., followed by labeling with Texas Red conjugated goat anti-mouse and Alexa Fluor-488 donkey anti-rabbit antibodies (ThermoFisher Scientific). To assess the ER localization of biotinylated proteins, cells were probed with a rabbit antibody against calnexin (Cell Signaling, C5C9) and Alexa Fluor 488 streptavidin overnight at 4° C., followed by labeling with a Texas Red goat anti-rabbit antibody.

For the in vivo localization of ER-BioID^{HA}, primary endothelial cells were isolated from the lungs of secretome^{ENDO} mice or control (VE-cadherin Cre only) mice. Briefly, two mouse lungs per genotype were digested with type I collagenase and plated on gelatin, fibronectin, and collagen-coated flasks. The cells were then subject to sequential negative sorting by magnetic beads coated with a sheep anti-rat antibody using a Fc blocker (rat anti-mouse CD16/CD32, BD Pharmingen Catalog No. 553142) to remove macrophages and positive sorting by magnetic beads using an anti-intermolecular adhesion molecule 2 (ICAM2 or CD102) antibody (BD Pharmingen Catalog No. 553326) to isolate ECs (ICAM2 positive cells). These primary ECs were used within five passages after isolation for immunostaining. Similarly, to confirm the ER localization of ER-BioID^{HA}, the fixed cells were probed with a mouse antibody against the HA-Tag (Santa Cruz Biotechnology, sc-7392) and a rabbit antibody directed against calnexin (EMD Millipore Catalog No. AB2301) overnight at 4° C., followed by labeling with a goat anti-mouse Texas Red and Alexa Fluor-488 donkey anti-rabbit antibody (ThermoFisher Scientific). To verify the ER localization of biotinylated proteins, cells were probed with a rabbit antibody against calnexin (EMD Millipore Catalog No. AB2301) and Alexa Fluor 647 streptavidin overnight at 4° C., followed by labeling with an Alexa Fluor-488 donkey anti-rabbit antibody.

### Generation of Tissue-Specific Secretome Transgenic Mice

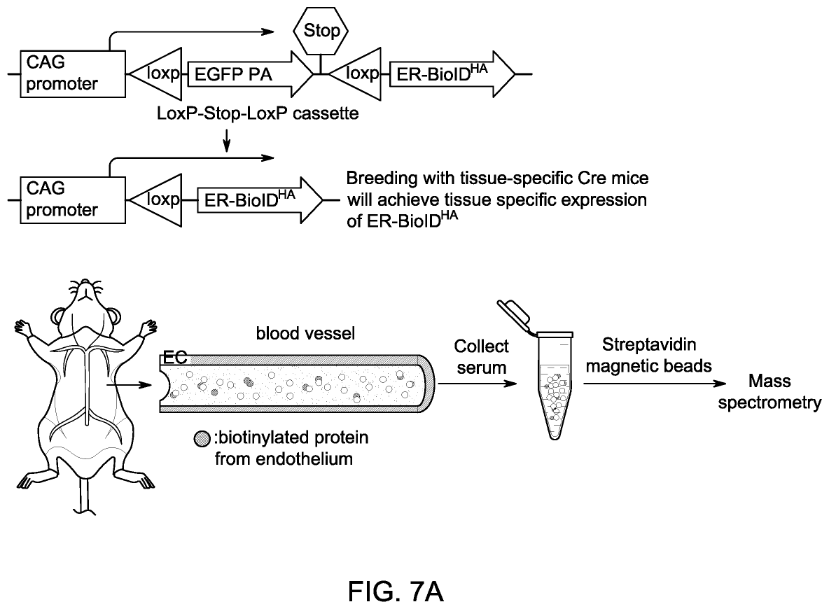

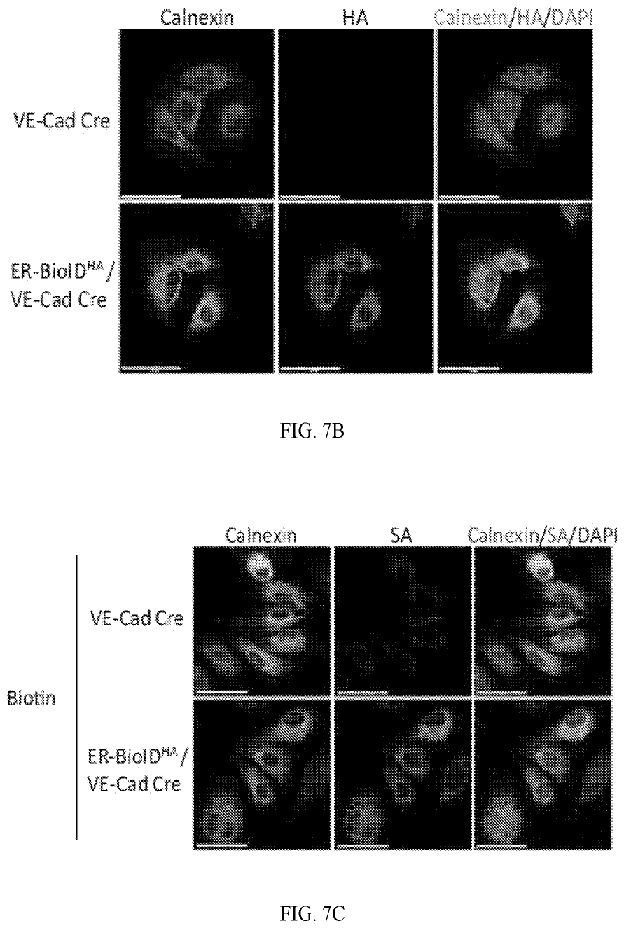

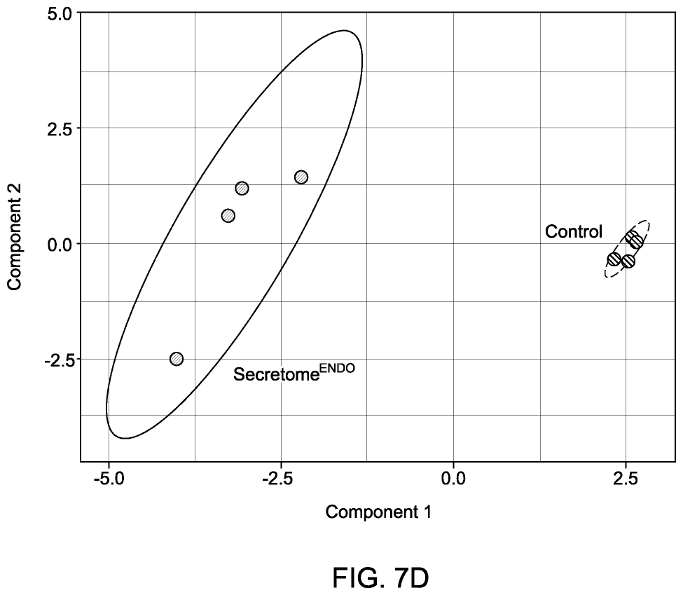

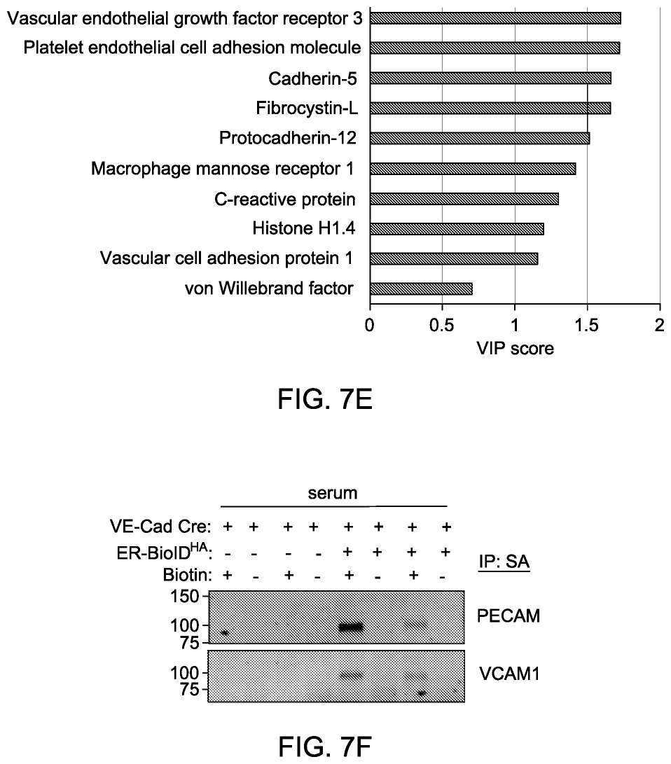

The ER-BioID^{HA} coding fragment was cloned into EcoRI/Nhel sites of the pCLE vector, resulting in a transgenic vector pCLE-ER-BioID^{HA} in which the ER-BioID^{HA} coding sequence was under the control of the CAG promoter (PCAG, a combination of the cytomegalovirus early enhancer element and chicken (3-actin promoter). A loxP-Stop-loxP (LSL) cassette (a DNA fragment containing an EGFP coding sequence followed by a poly[A] terminator) was placed between PCAG and BioID2-ERHA (FIG. 7A). After removing the nonrelevant sequences using Xho I and Dra III digestion, the transgenic vector was microinjected into the pronuclei of fertilized eggs to generate a transgenic mice (C57BL/6 background). The founder lines were genotyped by PCR analyses using the primers listed Table 2. These animals do not express the ER-BioID^{HA} transgene until crossed with cell-type-specific Cre-transgenic mice to remove the LSL cassette and place PCAG adjacent to the ER-BioID^{HA} coding sequence (secretome mouse; FIG. 7A). Thus, endothelial-specific-expressing mice, secretome^{ENDO} mice, were obtained by breeding the secretome mouse with Cdh5-Cre (Cre-recombinase under the VE-cadherin promoter) mice (The Jackson Laboratory no. 006137) to delete the LSL cassette only in endothelial cells. Secretome^{MUSCLE} mice were generated similarly using the corresponding muscle-specific MCK-Cre mice (The Jackson Laboratory no. 006475).

### Animal Studies

Except when noted, an equal number of male and female mice 8-12-wk-old mice were used in this analysis. For biotin labeling of the in vivo secretome, prior to serum collection, the mice were administered both i.p. and subcutaneously with 500 μL of a 2-mg/mL biotin solution (Sigma-Aldrich) once a day for five consecutive days. Except when noted, the animal's chow was also soaked in a solution of 2-mg/mL biotin (2-mg biotin per gram of chow) for five consecutive days before harvest. The sera were collected after terminally bleeding. Approximately 300-μL serum from each mouse was incubated with 100-μL streptavidin beads overnight. The beads were collected using a magnetic stand, and the biotinylated proteins were collected in sodium dodecyl sulfate (SDS) loading buffer and analyzed by mass spectrometry. For the exercise paradigm, mice were provided with InnoWheel on top of an InnoDome for a month. In each cage, there was one apparatus for every two mice. Serum myostatin levels were assessed in a separate set of 3-month-old wild-type male C57BL/6 mice either exercised (n=15) or nonexercised mice (n=8) using a GDF-8/Myostatin Quantikine ELISA Kit (R&D Systems, DGDF80). For confirmation of endothelial biotinylated proteins, 200 μL of serum per mouse (n=4 per genotype) were incubated with streptavidin beads overnight. The beads were collected using a magnetic stand, and the biotinylated proteins were collected in SDS loading buffer and analyzed by Western blotting using an anti-PECAM antibody (Abcam, ab222783) or an anti-VCAM-1 antibody (Abcam, ab134047). For assessing whether ER-BioID^{HA} was secreted into the circulation, 20 μL of serum from either secretome^{MUSCLE} mice or control mice were analyzed by Western blotting using an anti-HA-tag antibody.

### Mass Spectrometry

Each sample was heated at 85° C. for 5 minutes and separated on a 10% Bis-Tris Novex mini-gel (Invitrogen) using the MES buffer system. The gel was stained with coomassie and each lane was excised into ten equally sized segments. Gel segments were reduced using dithiothreitol, alkylated with iodoacetamide and then subjected to digestion with trypsin (Promega, Madison, Wis.). Digests were analyzed by nano LC/MS/MS with a NanoAcquity HPLC system (Waters, Milford, Mass.) interfaced to a Fusion Lumos tandem mass spectrometer (ThermoFisher, San Jose, Calif.). Peptides were loaded on a trapping column and eluted over a 75 μm analytical column at 350 nL/minute; both columns were packed with Luna C18 resin (Phenomenex, Torrance, Calif.). A 30 minute gradient was employed for each segment. The mass spectrometer was operated in data-dependent mode, with MS and MS/MS performed in the Orbitrap at 60,000 FWHM resolution and 15,000 FWHM resolution, respectively. APD was turned on. The instrument was run with a 3 second cycle for MS and MS/MS. Dynamic exclusion and repeat settings ensured each ion was selected only once and excluded for 30 seconds thereafter. Product ion data were searched against the combined forward and reverse Swissprot H. sapiens protein database using a locally stored copy of the Mascot search engine v2.6 (Matrix Science, London, U.K.) via Mascot Daemon v2.6. Peak lists were generated using the Proteome Discoverer v2.1 (ThermoFisher). The database was appended with common background proteins. Search parameters were precursor mass tolerance 10 ppm, product ion mass tolerance 0.02 Da, 2 missed cleavages allowed, fully tryptic peptides only, fixed modification of carbamidomethyl cysteine, variable modifications of oxidized methionine, protein N-terminal acetylation and pyro-glutamic acid on N-terminal glutamine. Mascot search result flat files (DAT) were parsed to the Scaffold software v4.8 (Proteome Software); data were filtered 1% protein and peptide level false discovery rate (FDR) and requiring at least two unique peptides per protein. All mass spectroscopy was performed by MS Bioworks (Ann Arbor, Mich. USA).

### Statistical Analyses

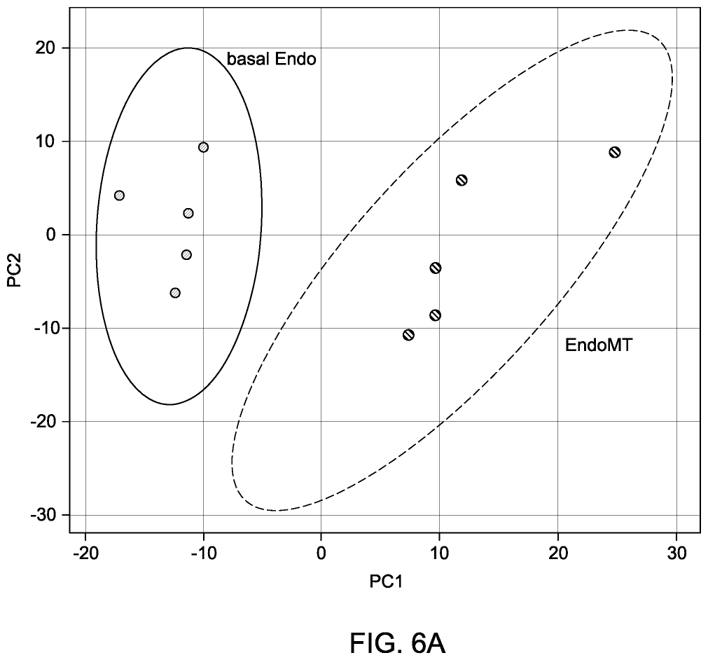

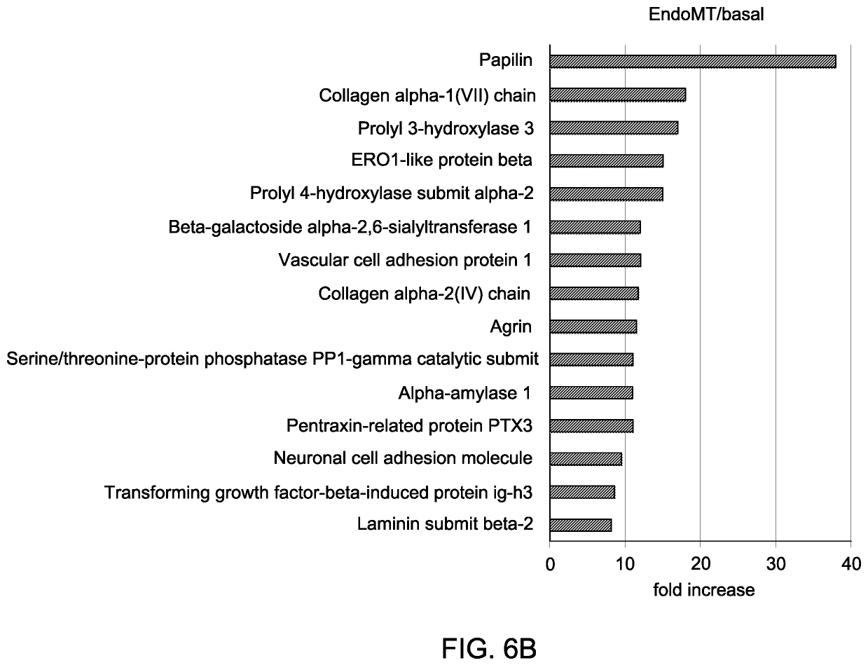

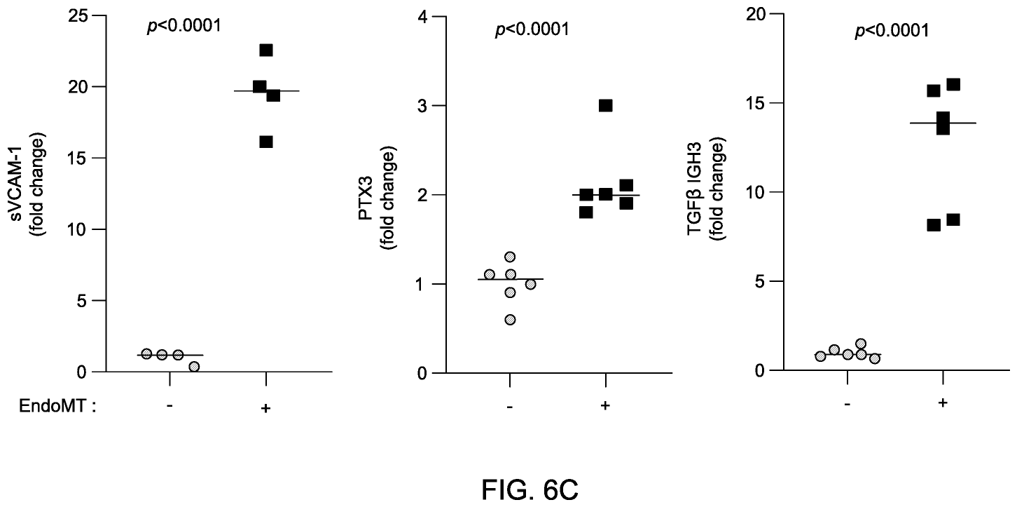

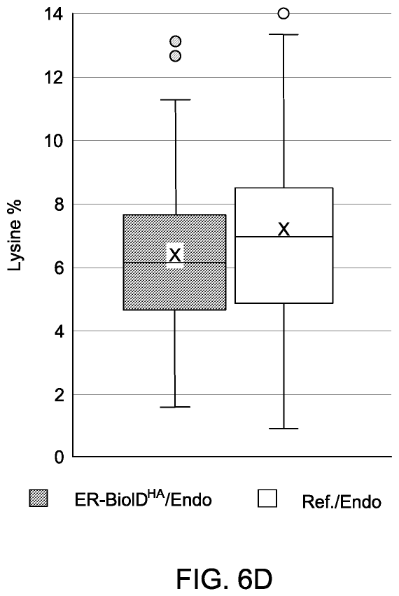

Principal component analysis (PCA) was performed on the in vitro mass spectroscopy data. PCA is a multivariate modelling and analysis technique commonly used to identify patterns in data and to emphasize samples similarities and differences by reducing the number of dimensions based on their similarities and differences. PCA analysis was performed on the technical replicates extracted from the mass spectroscopy data sets. The first 2 PCs (PC1 and PC2), accounted for the highest variability of the data, are illustrated in PCA plot (FIG. 6A) indicating variance in the data among test and control samples.

For the in vivo analysis of the Secretome mice, PLS (Partial Least Squares) regression, a method for relating two blocks of variables, X (proteins) and Y (samples), to each other via a linear multivariate model, was employed. PLS has many advantages over more traditional regression methods such as handling noise, missing data, colinearity among variables, and large number of variables (proteins) than samples. PLS-DA is a variant of standard PLS regression in which the block of Y-variables (samples) consists of a set of binary indicator variables (one for each class) denoting class membership. PLS-DA, therefore, attempts to separate the classes based on a training set of samples with known class membership and construct variables in such a way that a maximum separation is obtained among them. PLS-DA can be very useful in addition to PCA to correlate variation in a dataset with class membership and to select important variables, called VIP (variable importance), involved in class distinction. To improve the specificity of analysis two filtering rules were instituted. First, the protein had to be found in >50% of the experimental samples and the number of peptides in the experimental group has to average >4 peptides (total peptide counts for a given protein/total number of mice). Similarly, if the protein was found in >50% of control samples and the average peptides in the controls was >4 that protein was excluded. The VIP identifies those variables that are important for explaining the variance in the classification model. The VIP coefficient of a protein is calculated as a weighed sum of the squared correlations between the PLS-DA components and the original variable.

GraphPad Prism was used for statistical analyses. The two-tailed unpaired Student's t test was used. P<0.05 was considered as a significant difference. In comparisons between two groups with equal variance, unpaired two-tailed Student's t-tests was used to identify significant (P<0.05) differences.

### Data Availability

All study data are included in the article and supporting information. The raw proteomic data are deposited in the ProteomeXchange repository of Proteome Central with the identifier PXD022694.

### Results

### Determining the In Vitro Secretome Using Proximity Biotinylation

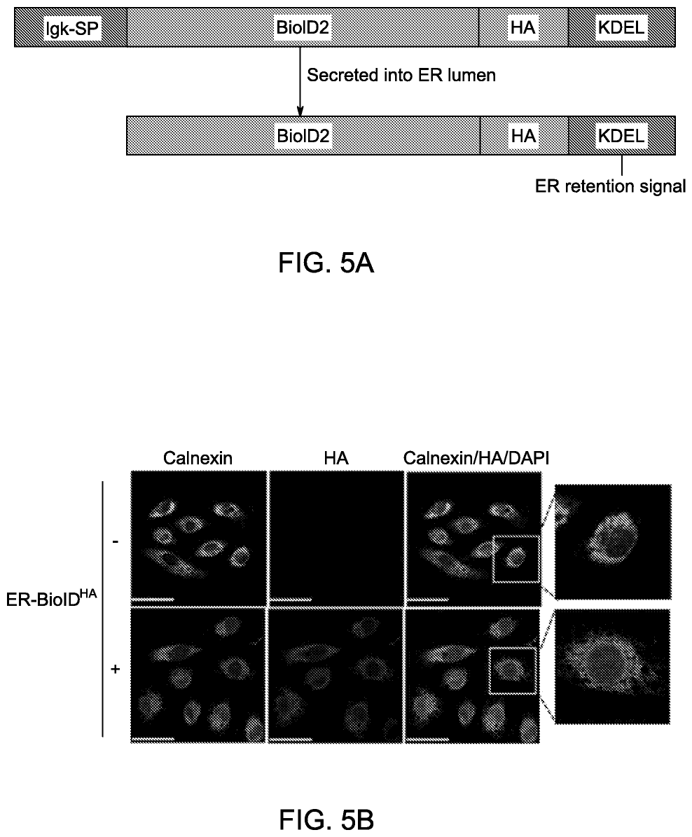

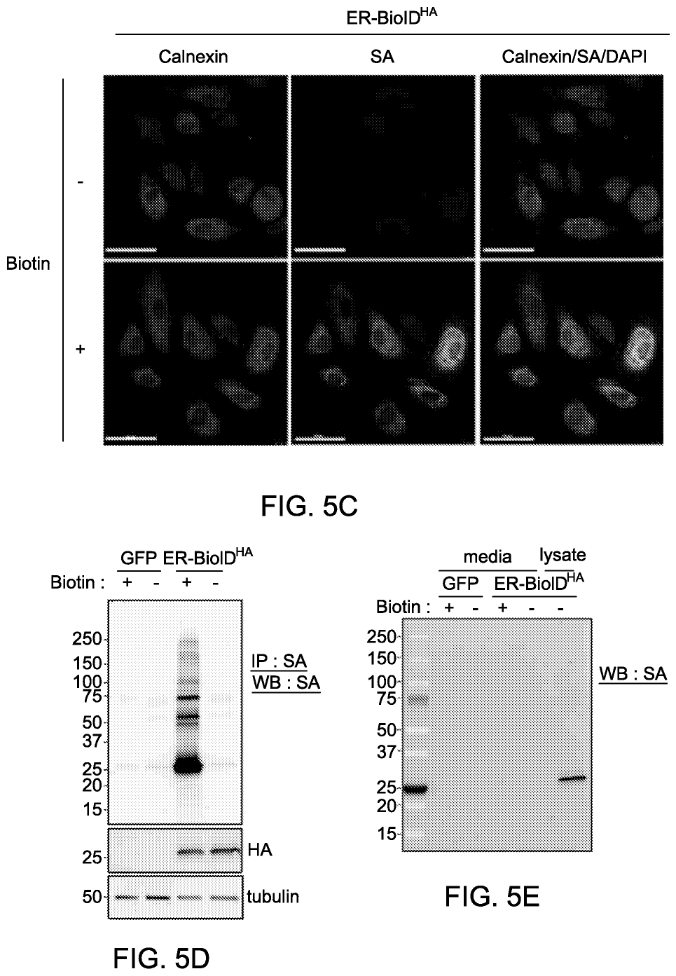

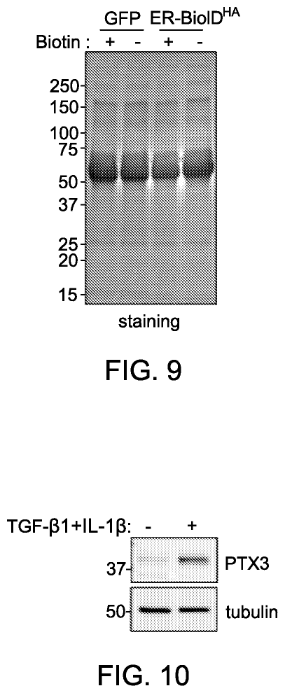

In an effort to tag and purify secreted proteins, ER-BioID^{HA}containing the promiscuous biotinylation enzyme BioID2 in frame with an HA-epitope tag and the KDEL peptide C-terminal ER retention sequence was constructed (FIG. 5A). To allow the expression of ER-BioID^{HA} in a wide variety of cell types, this construct was incorporated into a lentiviral vector. Subsequent lentiviral-mediated delivery to primary endothelial cells in culture demonstrated ER-BioID^{HA} extensively colocalized with the ER resident protein calnexin (FIG. 5B). In cells expressing ER-BioID^{HA}, the addition of exogenous biotin (50 μM) resulted in the accumulation of intracellular biotinylated proteins as evidenced by streptavidin-dependent immunohistochemistry (FIG. 5C). This accumulation of biotinylated proteins was not evident when the culture medium was not supplemented with exogenous biotin (FIG. 5C). Given that ER-BioIDHA localizes to the ER lumen, it was evaluated whether these intracellular biotinylated proteins would eventually appear in the conditioned medium of ER-BioIDHA-expressing cells. Streptavidin purified supernatants of primary endothelial cells expressing either ER-BioIDHA or a green fluorescent protein (GFP) control demonstrated that in the presence of exogenous biotin, only endothelial cells expressing ER-BioID^{HA} secreted multiple biotinylated proteins into the conditioned medium (FIG. 5D and FIG. 9). ER-BioID^{HA} itself was not observed in the culture medium, suggesting that biotinylation occurred intracellularly (FIG. 5E).

The detection of the secretome of cells in culture is often complicated by abundant serum proteins present in culture medium. While it is theoretically possible to perform these types of experiments in serum-free conditions, this is not a viable strategy for many cell types or for experiments that require extended periods of time in culture. In contrast, ER-BioID^{HA}-expressing cells allow one to rapidly purify secreted proteins using streptavidin thereby largely obviating the concerns about abundant serum proteins (e.g., albumin) present in standard culture medium. To test the utility of this strategy, the conditioned medium of ER-BioID^{HA}-expressing endothelial cells was analyzed under basal conditions and 3 days after being stimulated with TGF-β1 and IL-1β. A large number of secreted proteins were identified under both basal and EndoMT conditions. Principal component analysis (PCA) demonstrated that these two cell states could be readily distinguished by their secretome (FIG. 6A). A number of specific proteins whose abundance in the culture medium markedly increased following induction of EndoMT were noted (FIG. 6B). Included in the list of secreted proteins are proteins that might be expected to increase with a transition to a more mesenchymal phenotype including the extracellular matrix protein papilin as well as collagen Type 8 and related proteins (e.g., COL8A1, P3H3, and P4HA2). The overall fidelity of this approach was assessed by directly analyzing the expression of three proteins identified by the ER-BioIDHA approach as being differentially secreted. As seen in FIG. 6C, traditional ELISA-based strategies confirmed the ER-BioIDHA approach by demonstrating that endothelial cells induced to undergo EndoMT significantly increased their secretion of transforming growth factor-β-induced protein immunoglobulin G (IG)-H3 as well as increasing their secretion of pentraxin 3, a protein previously associated with EndoMT transition. It was also observed that a similar increase could be observed in cell lysates of endothelial cells stimulated to undergo EndoMT (FIG. 10). This analysis also demonstrated that EndoMT was accompanied by the increased abundance of the cleaved soluble fragment of the VCAM-1 receptor in the culture medium. Soluble VCAM-1 is normally generated by endothelial metalloproteinase cleavage of the extracellular portion of the transmembrane receptor. These results suggest that the conversion from an EndoMT phenotype likely involves accelerated removal of endothelial-specific proteins, such as VCAM-1 through an EndoMT-stimulated posttranslational cleavage event.

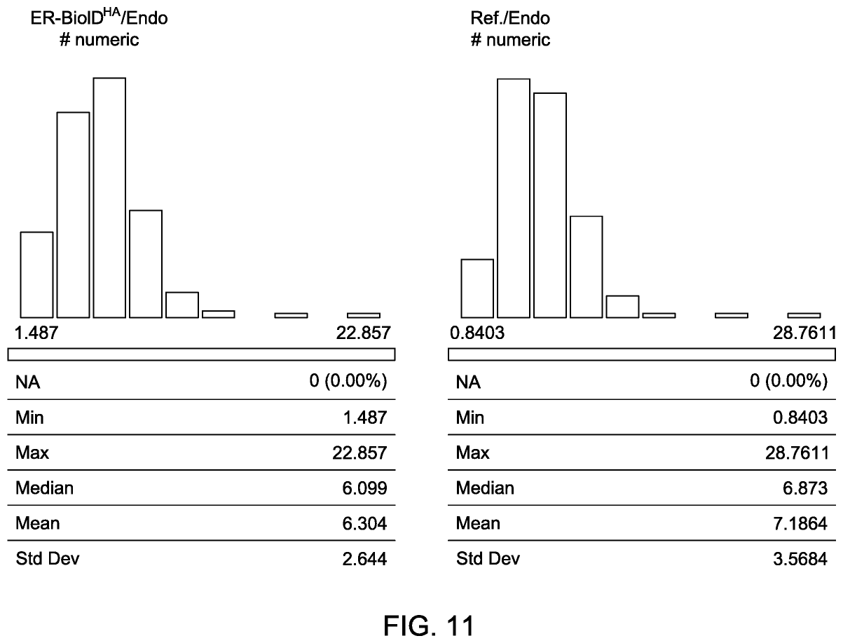

Finally, given that biotinylation occurs on lysine residues, whether the ER-BioID^{HA}—based strategy would preferentially label secreted proteins with high lysine content was evaluated. the lysine content of a reference data set (Tunica et al., Proteomics, 9:4991-4996 (2009)) and the lysine content of the top 182 endothelial proteins detected using the ER-BioID^{HA}—based approach were compared. This analysis suggested both sets of proteins had similar lysine content (FIG. 6D and FIG. 11). As such, this ER-BioID^{HA}—based strategy appears suitable for the rapid identification and characterization of the in vitro secretome of a wide variety of cell types under various experimental conditions.

### Generation of the Secretome Mouse

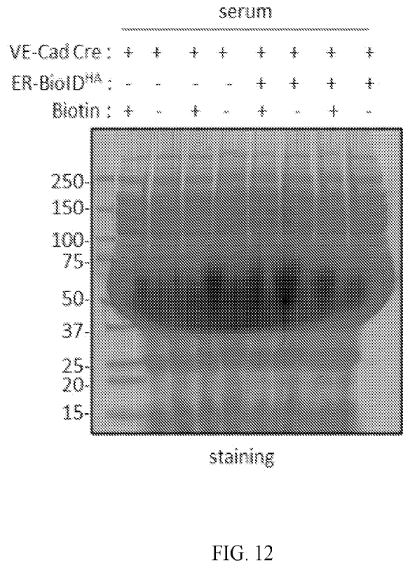

ER-BioID^{HA} expression can also provide an in vivo method for detecting protein secretion. A transgenic mouse line was generated in which ER-BioID^{HA} expression was initiated by excision of a floxed enhanced GFP (EGFP) cassette (FIG. 7A). Crossing this line with a tissue-specific Cre-recombinase allows for cell or tissue-restricted deletion of EGFP and the corresponding expression of ERBioID^{HA}. A mouse line for endothelialspecific expression of ER-BioID^{HA} was generated by crossing the transgenic line to mice expressing vascular endothelial (VE)-cadherin Cre, hereafter termed secretome^{ENDO}. To confirm the correct subcellular in vivo targeting of the transgene endothelial cells from the experimental secretome^{ENDO} mice or from corresponding control animals (VE-cadherin Cre only mice) were isolated. The ER-restricted HA-tagged transgene was readily detectable only in endothelial cells derived from the experimental mice (FIG. 7B). Similarly, only these endothelial cells generated intracellular biotinylated proteins when exposed to exogenous biotin (FIG. 7C). Serum from secretome^{ENDO} mice supplemented with biotin or from a similarly treated group of control mice that were positive for either the floxed ER-BioID^{HA} transgene or a Cre-recombinase transgene but not both constructs was obtained. Biotinylated proteins were purified using streptavidin and then analyzed by mass spectroscopy. As noted in FIG. 7D, using sPLSDA control mice could be readily distinguish from the experimental animals. Purified proteins with the greatest variable importance in projection (VIP) coefficients were subsequently identified (FIG. 7E). This list included well-known proteins previously identified as deriving from the endothelium included the endothelial cell surface receptors (e.g., VEGFR-3, VCAM1, PECAM1, and cadherin-5) as well as the von Willebrand factor. However, proteins not commonly associated with the endothelium, such as fibrocystin (Pkhd111) were also identified. C-reactive protein, a well-known modulator of endothelial function, was also detected. To provide additional confirmation of the mass spectroscopy results Western blot analysis of purified serum proteins derived from control and experimental animals with and without biotin supplementation was performed. These results supported the generation of circulating biotinylated proteins in the experimental mice only in the context of biotin supplementation (FIG. 7F and FIG. 12).

### Analyzing Muscle Secretion In Vivo

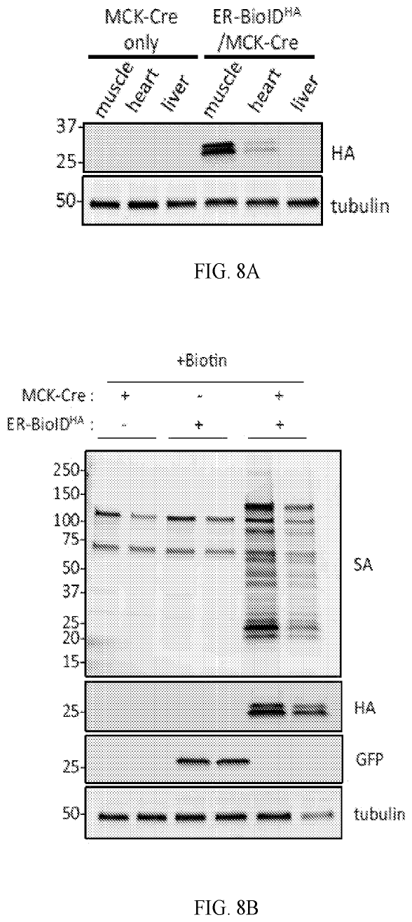

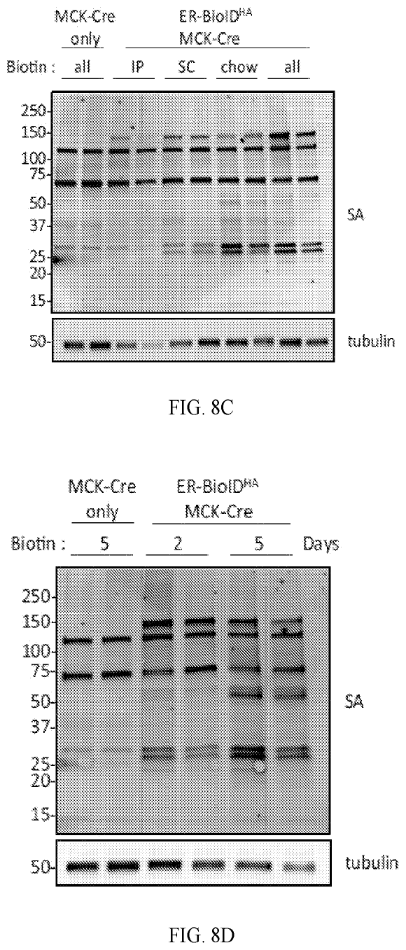

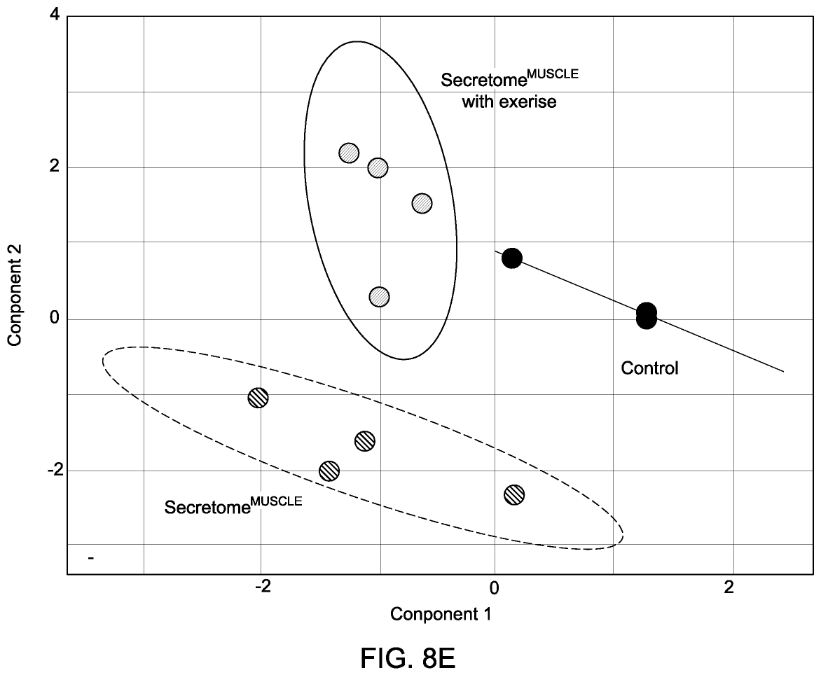

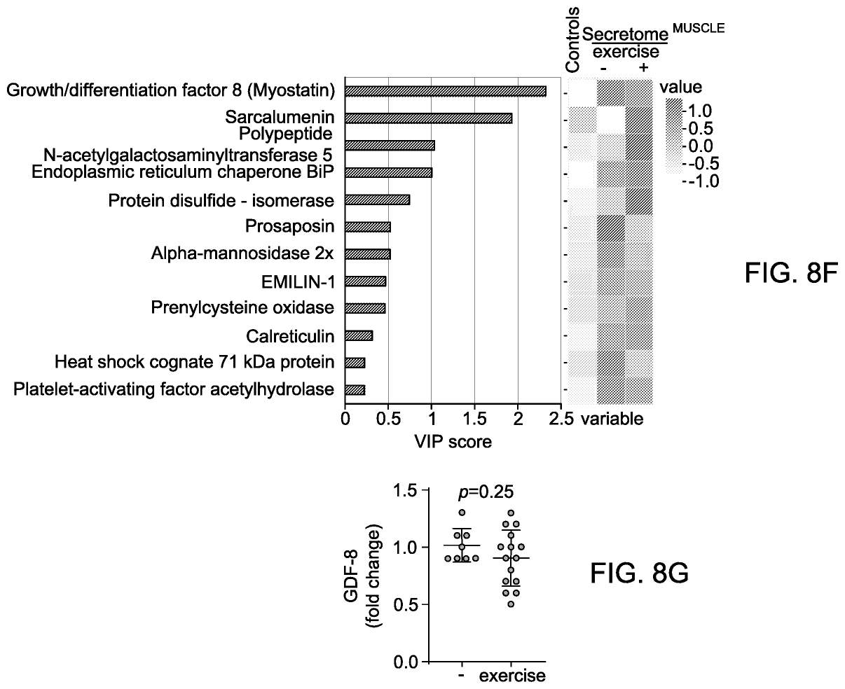

The transgenic line was crossed with mice expressing muscle creatine kinase (MCK)-Cre leading to ERBioID^{HA} expression in skeletal muscle and to a lesser extent in cardiac muscle but not in other tissues (FIG. 8A). Analysis of skeletal muscle cell lysate demonstrated that in the presence of biotin supplementation, ER-BioID^{HA} /MCK-Cre⁺ mice (hereafter denoted as secretome^{MUSCLE}) had multiple biotinylated proteins not evident in the skeletal muscle of the control animals expressing only MCK-Cre or ER-BioID^{HA} (FIG. 8B). Biotin supplementation in these animals came from a combination of multiple routes of administration (intraperitoneal, subcutaneous, and oral feeding) over a 5-day period where a combination approach appeared to increase labeling efficiency (FIG. 8C). It was also noted that biotin supplementation for 5 days was superior to supplementing mice for just 2 days (FIG. 8D).

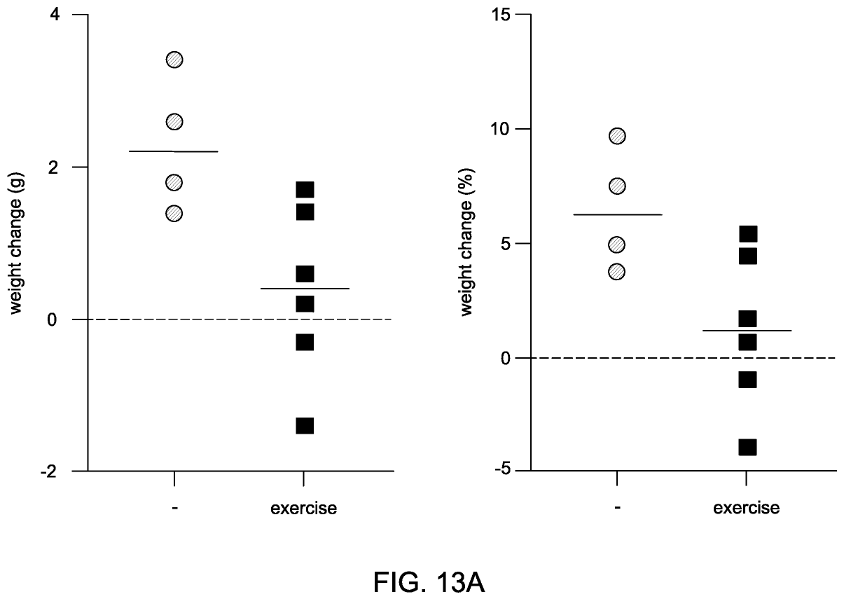

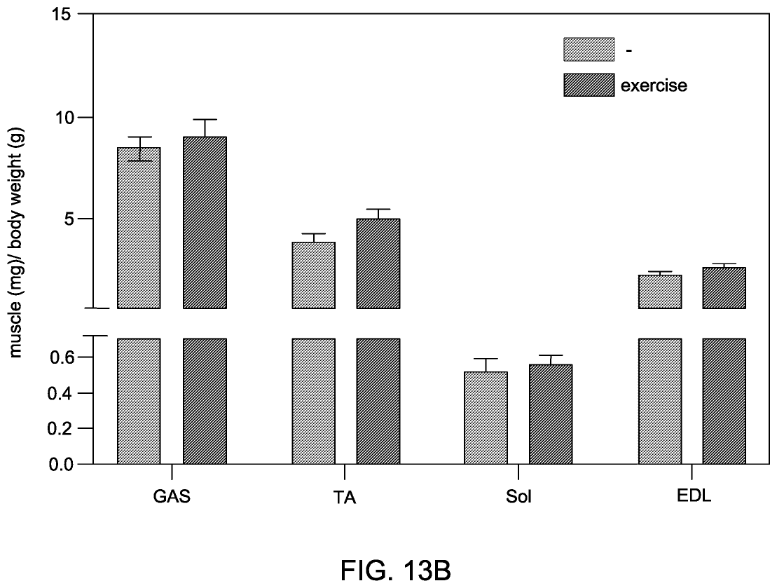

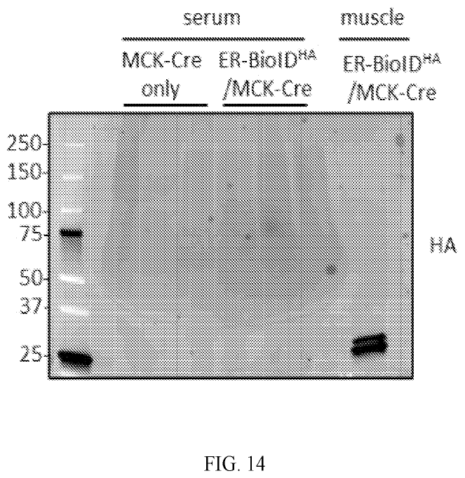

Secretome^{MUSCLE} mice were separated into cages with or without a running wheel in order to understand how the muscle-derived secretome would change with voluntary exercise. This voluntary wheel running was of sufficient intensity to effect body weight but not to induce skeletal muscle hypertrophy (FIG. 13). The secretome of secretome^{MUSCLE} mice with and without exercise were then compared. The serum of these experimental animals and the control animals that only expressed a Cre-recombinase transgene were isolated by streptavidin purification followed by mass spectroscopy. As was observed for cells in culture, ER-BioID^{HA} itself was not detectable in the serum, suggesting the biotinylation enzyme is not directly secreted into the circulation (FIG. 14). As noted in FIG. 8E, sPLSDA was capable of delineating secretome^{MUSCLE} mice with and without exercise from each other and from control mice based on their purified secretome. Purified proteins with the greatest VIP coefficients were subsequently identified (FIG. 8F). The VIP proteins identified from the secretome^{MUSCLE} mice exhibited no overlap with the proteins previously identified from the secretome^{ENDO} mice.

A number of the serum proteins identified as deriving from muscle altered their abundance with exercise (FIG. 8F). Among these was the muscle protein myostatin, whose abundance modestly decreased with exercise (average 29 peptide counts basally (n=4 mice), average 26 peptide counts with exercise (n=4 mice), and average of two peptide counts in the control group (n=7 mice)). To confirm these observations, a separate group of wild-type mice were subjected to similar cage conditions with or without the option for exercise. Analysis of the serum of these animals revealed a similar modest trend of reduced circulating myostatin in the exercise cohort (FIG. 8G).

### Example 3: Transcriptional and Proteomic Characterization of Telomere-Induced Senescence in a Human Alveolar Epithelial Cell Line

This example describes using a secretome mouse to identify secretome changes that potentially contribute to the pathogenesis of idiopathic pulmonary fibrosis (IPF).

### Methods

### Tissue Culture and Generation of Stable Cell Lines

A549 cells were acquired from ATCC and cultured in DMEM supplemented with 10% fetal bovine serum and penicillin (120 U/mL), streptomycin (100 mcg/mL), and L-glutamine (2 mM). A construct for conditional induction of telomere dysfunction was generated by cloning a truncated version of human TRF2 protein that lacks the N-terminal basic domain and C-terminal Myb domains into the lentiviral vector pCW57-GFP-2A-MCS. Lentiviral particles were generated and used to transduce low-passage A549 cells. Following transduction, individual clones of cells were selected that showed strong expression of the transgene in the presence of 2 μg/mL doxycycline. Proliferation studies were carried out by plating three independent cultures of each cell line and enumerating cells at each passage. The total number of cells were log2 transformed and plotted against time. Fresh doxycycline (2 μg/mL final concentration) was added at each passage. Clonogenic assays were performed by plating 1,000 cells in 10 cm dishes and enumerating colonies following staining with crystal violet after 12 days in culture. Media was replaced with fresh doxycycline (2 μg/mL final concentration) every 48 hours during the course of the experiment. Senescence-associated beta-galactosidase (SA-βgal) was stained according to manufacturer's protocol (Cell Signaling Technologies). Proximity ligation experiments were carried out by expressing a modified biotin ligase (BioID2) that had been targeted to the endoplasmic reticulum (ER) by addition of a N-terminal IgK signal sequence and C-terminal ER retention sequence (KDEL; SEQ ID NO:1).

### Western Blots and Immunoprecipitation

Western blots were performed following standard procedures and antibodies specific for Flag epitope (M2, Millipore Sigma), V5 (Thermo Fisher), HA (Millipore Sigma), p21 (Cell Signaling Technologies), and GAPDH (BioRad) were employed. Briefly, cells were lysed in RIPA buffer containing protease and phosphatase inhibitors (MiniComplete, Roche). Following protein quantitation, 20-40 μg of protein or 18 μL of media were separated under reducing conditions using SDS-PAGE and transferred to PVDF membranes. Proteins were blotted with antibodies specific for the desired protein and visualized on a ChemiDoc MP gel documentation system (BioRad). Immunoprecipitation of V5-tagged proteins was accomplished by incubating media containing V5-tagged proteins with Anti-V5 agarose (Millipore Sigma) according to the manufacturer's instructions.

### Transcriptional Profiling and Analysis

Total RNA was isolated from biologic replicates (n=3) of cultured cells using RNAeasy kits (Qiagen) according to manufacturer's protocol and sent for library preparation, sequencing, quality control, alignment, differential expression analysis, and preliminary enrichment analysis at Novogene (Sacramento, Calif.). Approximately 20 million paired-end fragments were sequenced for each sample. The raw data have been deposited in NCBI's Gene Expression Omnibus GSE155941. Expression data from senescent murine AEC2s were obtained from GSE56892. Additional enrichment analyses were conducted using Ingenuity Pathway Analysis (Qiagen), Gene Ontology (GO), and Kyoto Encyclopedia of Genes and Genomes (KEGG). Differential expression of several genes was confirmed using quantitative real-time PCR with primers specific for the selected genes.

### Proximity Labeling and Mass Spectrometry

Validation of the BioID2 targeting and function was accomplished by transfecting cells stably expressing ER-targeted BioID2 with a plasmid encoding V5-tagged human SFTPA2 cDNA (pCDNA3-V5-SFTPA2). Eighteen hours after transfection, media was supplemented with biotin (100 μM). The next day, cells and media were collected for western blot analysis. V5-tagged SFTPA2 was immunoprecipitated with anti-V5 resin (Millipore). Detection of biotinylated proteins was accomplished by incubating membranes with streptavidin conjugated to horseradish peroxidase (Strep-HRP) and developing the membranes according to the manufacture's protocol (Vector Laboratories). The unbiased proteomic screen of telomere dysfunction-induced senescence-related changes was carried out by comparing TRF2-DN-BioID2 and TRF2-DN-BioID2+Doxycline. Four days after addition of doxycycline, biotin was added to the media. Eight hours later, cells were washed to remove excess biotin and fresh media was added. Twenty-four hours later, the supernatant was collected, and biotinylated proteins were purified by incubating media with streptavidin coated beads according to the manufacturer's protocol (Dynabeads MyOne Streptavidin C1; Invitrogen). Half of the sample was eluted at 95° C. for 10 minutes in loading buffer and run on a 4-15% SDS-PAGE gel to evaluate yield of recovered protein. The remainder of the protein coated beads were sent to MS Bioworks (Ann Arbor, Mich.) for mass spectrometry analysis where they were eluted, gel separated, split into 10 samples based on molecular weight, and digested samples were analyzed by nano LC/MS/MS with a Waters NanoAcquity HPLC system interfaced with a ThermoFisher Q Exactive mass spectrometer. A single sample was submitted for each condition. Data were searched using Mascot (Matrix Science) and parsed into Scaffold™ (Proteome Software Inc.) for validation, filtering and to create a non-redundant list per sample. Data were filtered using a 1% protein and peptide level false discovery rate (FDR) and by requiring at least two unique peptides per protein.

### Immunostaining and Imaging

Cells were grown on coverslips and fixed in 2% PFA for 10 minutes. Following fixation cells were washed, permeabilized with Triton X-100, and blocked with goat serum. Coverslips were incubated with primary antibodies including rat anti-HA (Millipore Sigma) and rabbit anti-calnexin (Cell Signaling). Proteins were visualized with secondary antibodies conjugated to Alexa 594 and Alexa 647 (Thermo Fisher). Nuclei were stained with 4′,6-diamidino-2-phenylindole (DAPI). Images were obtained at the Center for Biologic Imaging at the University of Pittsburgh on an Olympus FluoView Confocal microscope. Brightfield photomicrographs were captured on an Observer A.1 (Zeiss) equipped with AxioCam MRc camera.

### Multiplex Screen of Serum Biomarkers

Transcriptional and proteomic data were used to rationally select 17 candidate biomarkers for evaluation in a discovery cohort of control (n=30) and IPF (n=50) patients. Plasma samples from these patients were evaluated using Luminex® panels purchased from R&D systems. Candidates biomarkers were selected based on their differential expression in this study, the availability of compatible commercial assays to simultaneously measure several proteins, and their dilution compatibility with the chosen assay. For this initial study, only proteins that had previously been reported to be detectable in human plasma samples were evaluated. Panels were analyzed on a Bio-Plex reader (Bio-Rad) according to the manufacturer's protocol. Biomarkers selected from the discovery round were evaluated for correlations with baseline pulmonary function studies in IPF patients.

### Human Subjects

Clinical, physiologic, and high-resolution computed tomography studies of these patients supported the diagnosis of IPF. Patients fulfilled the criteria of the American Thoracic Society and European Respiratory Society for the diagnosis of IPF at the time of diagnosis. Patients with known causes of interstitial lung disease were excluded. Control patients consisted of unrelated healthy subjects, and had no self-reported advanced lung diseases.

### Statistical Analysis

All cellular images shown are representative of multiple experiments. RNA-seq differential expression analysis was performed using the DESeq2 R package. Fisher's exact test was used for differential expression analysis of mass spectrometry identified proteins. Simple linear regression was used for differential protein vs. RNA correlation. Control vs. IPF plasma protein levels were evaluated using Welch's t-test of significance. The Benjamini-Hochberg procedure was used for all corrections of multiple testing. Pearson correlation coefficients for IPF patient baseline PFT values were calculated using square-root-transformed protein levels.

### Results

### Biotinylation of the Secretome

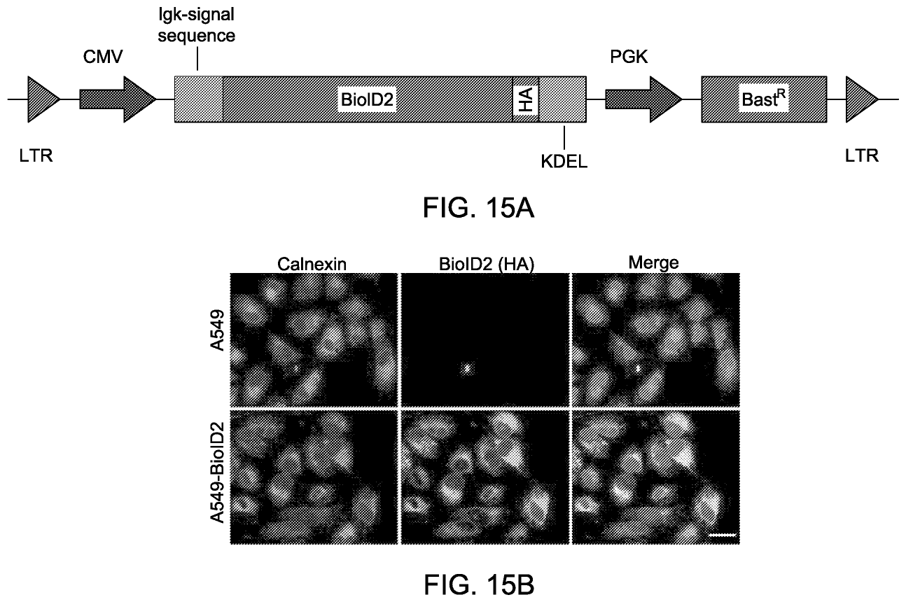

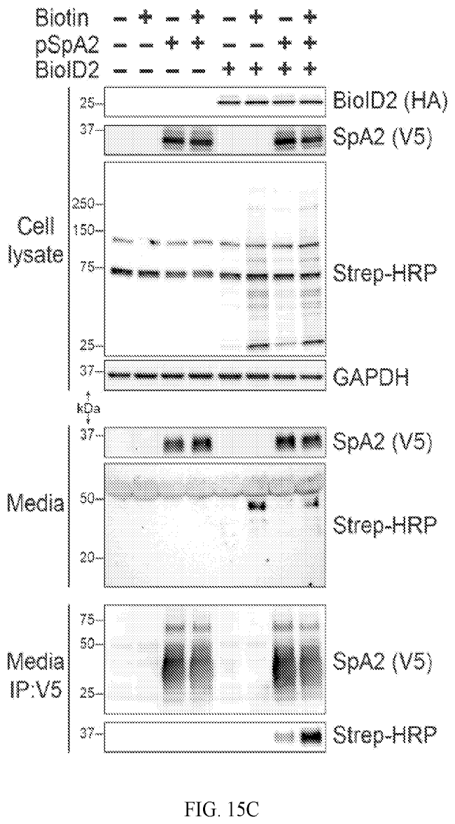

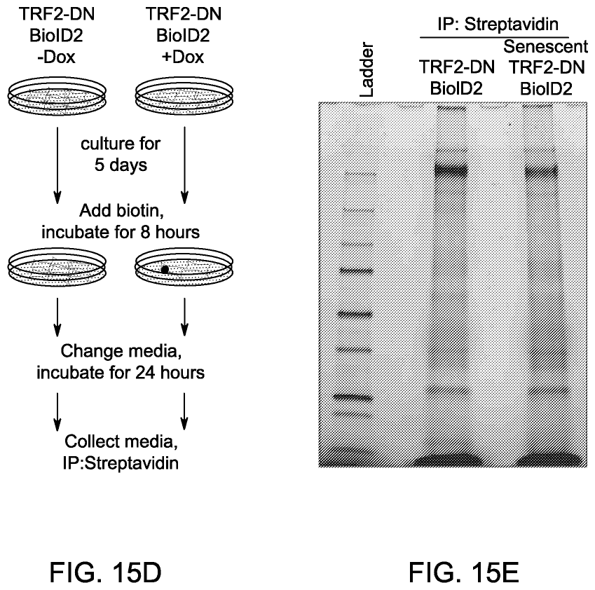

To identify senescence-associated changes in protein secretion in an unbiased manner, an endoplasmic reticulum (ER)-targeted biotin ligase (BioID2) capable of biotinylating proteins that traverse the classical secretion pathway was employed. A lentiviral vector system was used to stably express the ER-targeted BioID2 (FIG. 15A). Confocal microscopy confirmed ER-localization of the HA-tagged BioID2 (FIG. 15B). To test if the ER-targeted BioID2 system was functioning, a V5-tagged surfactant protein A2 (SpA2) plasmid that has been reported to be successfully secreted by A549 cells was introduced via transfection into the A549-BioID2 cell line. The presence of SpA2 in the transfected cell lysates was confirmed. An increase in biotinylation in lysates from the BioID2 line and a further increase in biotinylation in the lysates of these cells when cultured in the presence of excess biotin was verified. Upon blotting of the cell supernatants for the V5 epitope, SpA2 was readily detectable in the transfected lines (FIG. 15C, upper panels). The supernatants were probed with streptavidin-HRP and found an increase in biotinylated proteins in the BioID2 cell lines when grown in the presence of excess biotin (FIG. 3C, middle panels). Immunoprecipitation of SpA2 from supernatants followed by blotting with streptavidin-HRP demonstrated that SpA2 was being biotinylated uniquely in the A549-BioID2 system and to a greater extent when grown in the presence of excess biotin (FIG. 15C, lower panels).

### Senescence-Related Changes in the Secretome

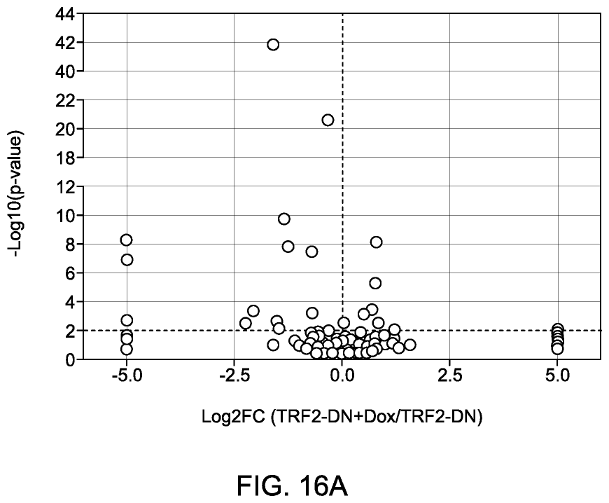

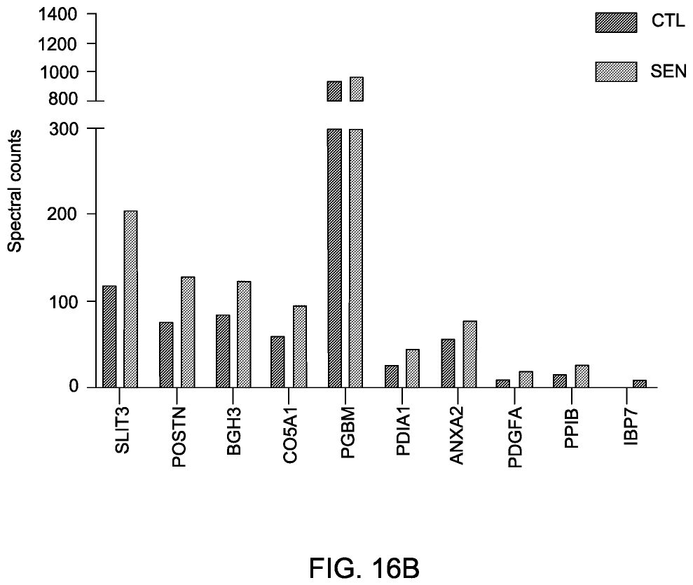

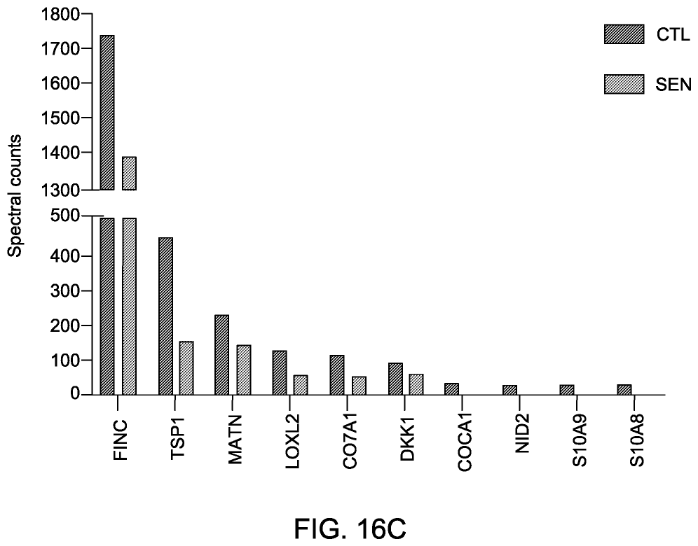

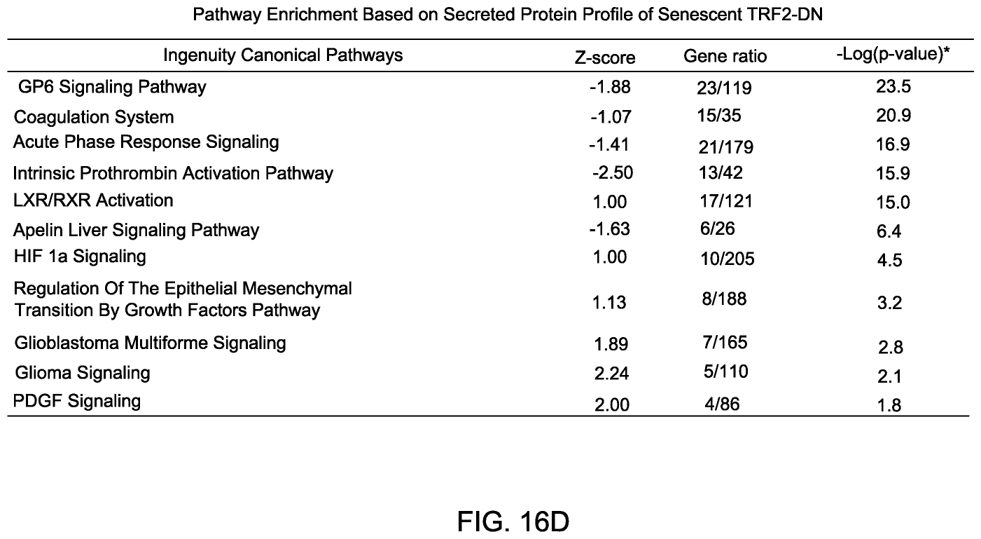

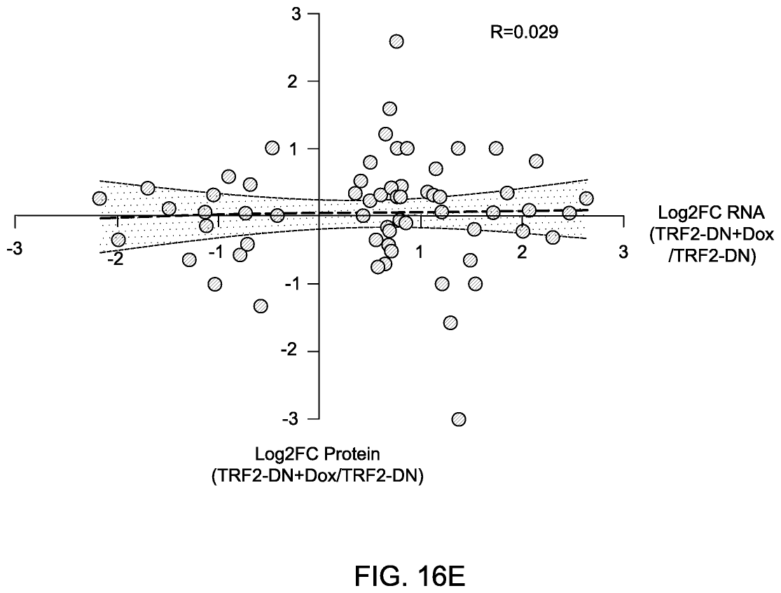

Cumulatively, 170 unique secreted proteins were identified by LC/MS/MS for the senescent and non-senescent groups (FIG. 16A). The most significantly upregulated proteins are shown in FIG. 16B, which includes the SASP protein, IBP7. Fibronectin 1 (FINC) and Thrombospondin 1 (TSP1) exhibited the greatest decrease in protein expression (FIG. 16C). Ingenuity Canonical Pathway analysis of the secretome revealed multiple significantly enriched pathways. These included pathways associated with coagulation, non-specific defense, adhesion, and lipid metabolism (FIG. 16D). The relationship between transcriptional fold change and corresponding proteomic fold change in the secretome and observed a poor correlation between the two datasets was analyzed (FIG. 16E).

### Example 4: Identification of Polypeptides Secreted From a Cultured Organ

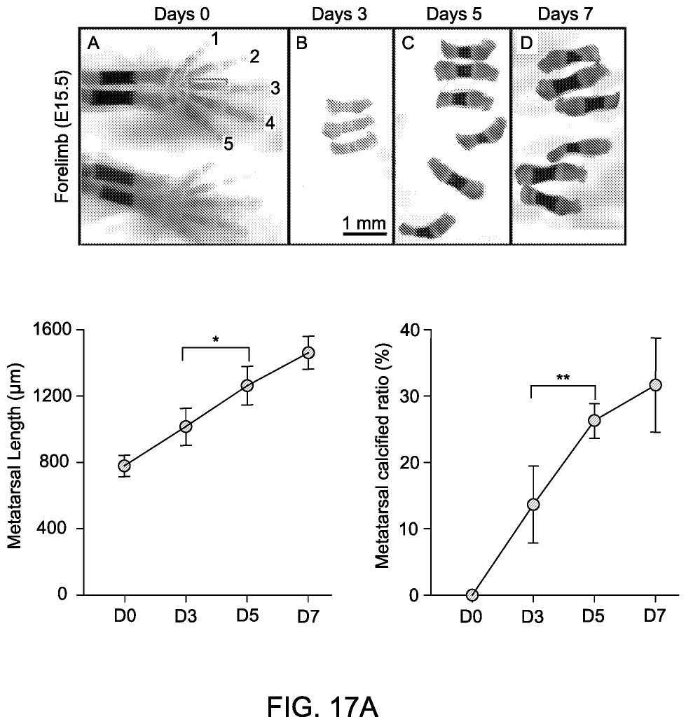

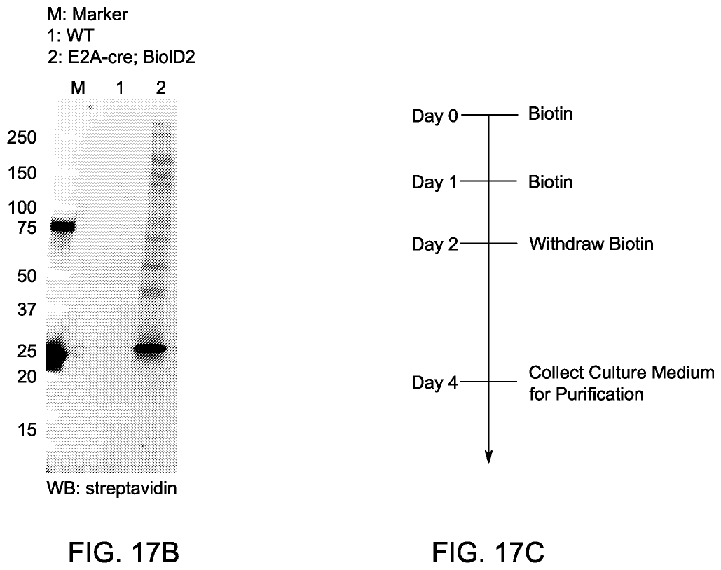

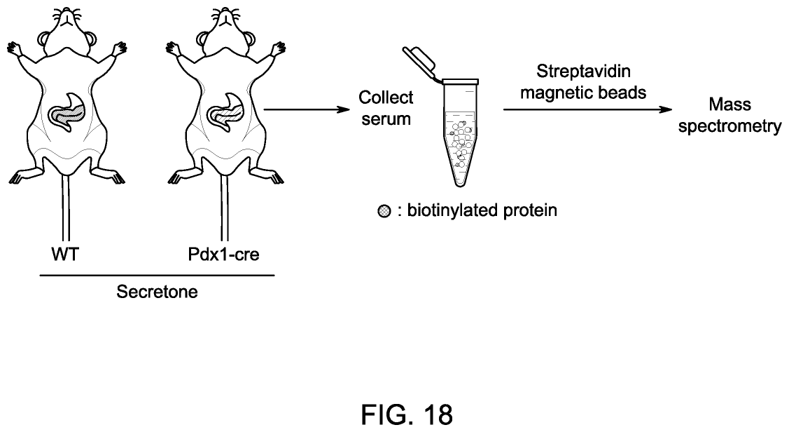

An exemplary method for identification of polypeptides secreted from a cultured organ is shown in FIG. 17.

Bone is harvested from a neonatal BioID2/E2A-cre transgenic mouse and maintained in culture with biotin. The biotin is removed from the culture on Day 2. Culture medium is collected on Day 4, and polypeptides secreted from the bone are identified.

### Example 5: Identification of Polypeptides Secreted From a Cancer

An exemplary method for identification of polypeptides secreted from pancreatic cancer is shown in FIG. 18.

A mouse containing Cre recombinase activity driven by the Pdx1 promoter, resulting in expression of Cre recombinase in the pancreas, is crossed with a mouse genetically pre-disposed to developing pancreatic cancer, such as those harboring floxed alleles of the oncogene K-RAS or a mutant form of p53. For example, a mouse genetically pre-disposed to developing pancreatic cancer can be a KPC strain mouse which is a tamoxifen-inducible model for pancreatic ductal adenocarcinoma (PDAC) carrying the Kras LSL G12D (Kras^{tm4Tyj}) allele, the Trp53^{tm1Brn} floxed allele, and the Tg(Pdxl-cre/Esrl*) Dam transgene. When crossed to the Secretome mouse, the addition of tamoxifen would activate mutant K-RAS and mutant P53, along with the expression of ER-BioID^{HA}, within the pancreas of the experimental mouse model. The serum of these animals contains biotinylated proteins secreted from the pancreas. Sampling at various time points after the addition of tamoxifen allows for the pancreatic-specific secretome of the normal pancreatic ductal cells (i.e. before tamoxifen addition) and at various time points along the progression of pancreatic ductal adenocarcinoma (e.g. pre-malignant, malignant, or invasive stage). These biotinylated proteins can be used as serum biomarkers for detecting PDAC at various stages of the disease.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A transgenic non-human animal, wherein the somatic cells of said non-human animal comprise nucleic acid comprising a nucleic acid sequence encoding a polypeptide comprising biotin ligase activity and an endoplasmic reticulum (ER) retention signal.

2. The non-human animal of claim 1, wherein said animal is a mouse.

3. The non-human animal of claim 1, wherein said ER retention signal is selected from the group consisting of KDEL (SEQ ID NO:1), SDEL (SEQ ID NO:6, PEEL (SEQ ID NO:7), and RDEL (SEQ ID NO:8).

4. The non-human animal of claim 1, wherein said biotin ligase is a bacterial BirA biotin ligase.

5. The non-human animal of claim 1, wherein said polypeptide comprises a peptide tag.

6. The non-human animal of claim 5, wherein said peptide tag is selected from the group consisting of an HA tag, a Myc-tag, a FLAG-tag a T7-tag, and a V5-tag.

7-9. (canceled)

10. The non-human animal of claim 1, wherein said nucleic acid comprises a promoter sequence followed by a recombinase recognition site followed by an intervening nucleic acid sequence followed by a recombinase recognition site followed by said nucleic acid sequence, wherein expression of said polypeptide does not occur unless a recombinase excises said intervening nucleic acid sequence via said recombinase recognition sites.

11. The non-human animal of claim 10, wherein said intervening nucleic acid sequence encodes one or more stop codons.

12. The non-human animal of claim 10, wherein said intervening nucleic acid sequence encodes an intervening polypeptide.

13. The non-human animal of claim 12, wherein said intervening polypeptide is an EGFP polypeptide.

14. The non-human animal of claim 1, wherein expression of said polypeptide within a somatic cell of said non-human animal results in secreted or membrane polypeptides of said somatic cell being biotinylated in the presence of biotin.

15. The non-human animal of claim 1, wherein said non-human animal is a non-human animal that consumed water, food, or both comprising biotin.

16-17. (canceled)

18. The non-human animal of claim 1, wherein the somatic cells of said non-human animal comprise a second nucleic acid, wherein said second nucleic acid comprises a nucleic acid sequence encoding a polypeptide comprising recombinase activity.

19. The non-human animal of claim 18, wherein said polypeptide comprising recombinase activity is a cre recombinase.

20. The non-human animal of claim 18, wherein said second nucleic acid comprises a tissue-specific promoter sequence operably linked to said nucleic acid sequence encoding said polypeptide comprising said recombinase activity.

21. The non-human animal of claim 20, wherein said tissue-specific promoter sequence is an endothelial cell-specific promoter sequence, a myocyte-specific promoter sequence, a pancreatic-specific promoter sequence, a kidney-specific promoter sequence, or an adipocyte-specific promoter sequence.

22-23. (canceled)

24. A method for making a transgenic non-human animal of claim 1, wherein said method comprises introducing said nucleic acid into a cell and allowing said cell to develop into said transgenic non-human animal.

25. The method of claim 24, wherein said cell is an egg cell or a cell of an embryo.

26. A method for making a transgenic non-human animal, wherein said method comprises mating a first mating partner with a second mating partner of the opposite sex, as compared to said first mating partner and of the same species as said first mating partner, to produce at least one offspring, wherein said first mating partner is a transgenic non-human animal of claim 1, and wherein the gamete of said first mating partner that results in said offspring comprises said nucleic acid.

27. A method for making a transgenic non-human animal, wherein said method comprises mating a first mating partner with a second mating partner of the opposite sex, as compared to said first mating partner and of the same species as said first mating partner, to produce at least one offspring, wherein said first mating partner is a first transgenic non-human animal, wherein the somatic cells and gametes of said first transgenic non-human animal comprise a first nucleic acid comprising a nucleic acid sequence encoding a polypeptide comprising biotin ligase activity and an endoplasmic reticulum (ER) retention signal, wherein said second mating partner is a second transgenic non-human animal, wherein the somatic cells and gametes of said second transgenic non-human animal comprise a second nucleic acid comprises a nucleic acid sequence encoding a polypeptide comprising recombinase activity, and wherein the gamete of said first mating partner that results in said offspring comprises said first nucleic acid.

28-34. (canceled)

35. A transgenic non-human animal, wherein the somatic cells of a cell type of said non-human animal comprise nucleic acid comprising a promoter sequence operably linked to a nucleic acid sequence encoding a polypeptide comprising biotin ligase activity and an endoplasmic reticulum (ER) retention signal, wherein said somatic cells of said cell type express said polypeptide.

36-56. (canceled)

57. A method for identifying a secreted or membrane polypeptide expressed by said cell type within a transgenic non-human animal of claim 35, wherein said transgenic non-human animal was treated with biotin, wherein said secreted or membrane polypeptide expressed by said cell type becomes biotinylated via said polypeptide, thereby forming a biotinylated polypeptide of said cell type, and wherein said method comprises:
(a) isolating said biotinylated polypeptide from a sample obtained from said transgenic non-human animal, and
(b) identifying said biotinylated polypeptide.

58-65. (canceled)


SEQUENCE LISTING (16 sequences)

SEQ ID NO: 1 - protein, 6 aa, artificial
  retention sequence
KDELER

SEQ ID NO: 2 - protein, 233 aa, artificial
  biotin ligase
MFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILTEEGIKEILSGEFSLRRS

SEQ ID NO: 3 - DNA, 701 bp, artificial
  nucleic acid encoding a biotin ligase
atgttcaagaacctgatctggctgaaggaggtggacagcacccaggagagactgaaggagtggaacgttagctacggcaccgccctggtggccgacagacagaccaagggcagaggcggcctgggcagaaagtggctgagccaggagggcggcctgtacttcagcttcctgctgaaccccaaggagttcgagaacctgctgcagctgcccctggtgctgggcctgagcgtgagcgaggccctggaggagatcaccgagatccccttcagcctgaagtggcccaacgacgtgtacttccaggagaagaaggtgagcggcgtgctgtgcgagctgagcaaggacaagctgatcgtgggcatcggcatcaacgtgaaccagagagagatccccgaggagatcaaggacagagccaccaccctgtacgagatcaccggcaaggactgggacagaaaggaggtgctgctgaaggtgctgaagagaatcagcgagaacctgaagaagttcaaggagaagagcttcaaggagttcaagggcaagatcgagagcaagatgctgtacctgggcgaggaggtgaagctgctgggcgagggcaagatcaccggcaagctggtgggcctgagcgagaagggcggcgccctgatcctgaccgaggagggcatcaaggagatcctgagcggcgagttcagcctgagaagaagctg

SEQ ID NO: 4 - protein, 236 aa, artificial
  polypeptide that includes an IgK signal peptide, a biotin ligase, an HA tag, and an ER retention signal
METDTLLLWVLLLWVPGSTGDFKNLIWLKEVDSTQERLKEWNVSYGTALVADRQTKGRGGLGRKWLSQEGGLYFSFLLNPKEFENLLQLPLVLGLSVSEALEEITEIPFSLKWPNDVYFQEKKVSGVLCELSKDKLIVGIGINVNQREIPEEIKDRATTLYEITGKDWDRKEVLLKVLKRISENLKKFKEKSFKEFKGKIESKMLYLGEEVKLLGEGKITGKLVGLSEKGGALILT

SEQ ID NO: 5 - DNA, 801 bp, artificial
  nucleic acid encoding a polypeptide that includes an IgK signal peptide, a biotin ligase, an HA tag, and an ER retention signal
atggagacagacacactcctgctatgggtactgctgctctgggttccaggttccactggtgacttcaagaacctgatctggctgaaggaggtggacagcacccaggagagactgaaggagtggaacgttagctacggcaccgccctggtggccgacagacagaccaagggcagaggcggcctgggcagaaagtggctgagccaggagggcggcctgtacttcagcttcctgctgaaccccaaggagttcgagaacctgctgcagctgcccctggtgctgggcctgagcgtgagcgaggccctggaggagatcaccgagatccccttcagcctgaagtggcccaacgacgtgtacttccaggagaagaaggtgagcggcgtgctgtgcgagctgagcaaggacaagctgatcgtgggcatcggcatcaacgtgaaccagagagagatccccgaggagatcaaggacagagccaccaccctgtacgagatcaccggcaaggactgggacagaaaggaggtgctgctgaaggtgctgaagagaatcagcgagaacctgaagaagttcaaggagaagagcttcaaggagttcaagggcaagatcgagagcaagatgctgtacctgggcgaggaggtgaagctgctgggcgagggcaagatcaccggcaagctggtgggcctgagcgagaagggcggcgccctgatcctgaccgaggagggcatcaaggagatcctgagcggcgagttcagcctgagaagaagctacccttacgatgtaccggattacgcaaaggacgagctgtga

SEQ ID NO: 6 - protein, 4 aa, artificial
  ER retention sequence
SDEL

SEQ ID NO: 7 - protein, 4 aa, artificial
  ER retention sequence
YEEL

SEQ ID NO: 8 - protein, 4 aa, artificial
  ER retention sequence
RDEL

SEQ ID NO: 9 - DNA, 21 bp, artificial
  primer
ctagagcctctgctaaccatg

SEQ ID NO: 10 - DNA, 26 bp, artificial
  primer
cagatcaggttcttgaagtcaccagt

SEQ ID NO: 11 - DNA, 20 bp, artificial
  primer
taagtctgaacccggtctgc

SEQ ID NO: 12 - DNA, 23 bp, artificial
  primer
gtgaaacagcattgctgtcactt

SEQ ID NO: 13 - DNA, 23 bp, artificial
  primer
gcggtctggcagtaaaaactatc

SEQ ID NO: 14 - DNA, 23 bp, artificial
  primer
gtgaaacagcattgctgtcactt

SEQ ID NO: 15 - DNA, 100 bp, artificial
  primer
tccgctagcatggagacagacacactcctgctatgggtactgctgctctgggttccaggttccactggtgacttcaagaacctgatctggctgaaggagg

SEQ ID NO: 16 - DNA, 56 bp, artificial
  primer
acttgtacactatcacagctcgtcctttgcgtaatccggtacatcgtaagggtagc